(19) United States
(12) Patent Application Publication
(10) Pub. No.: US 2011/0207789 A1
(43) Pub. Date: Aug. 25, 2011

(54) METHODS RELATED TO CASEIN KINASE II (CK2) INHIBITORS AND THE USE OF PURINOSOME-DISRUPTING CK2 INHIBITORS FOR ANTI-CANCER THERAPY AGENTS

(76) Inventors: Ye Fang, Painted Post, NY (US); Florence Verrier, Corning, NY (US)

(21) Appl. No.: 12/708,840

(22) Filed: Feb. 19, 2010

(51) Int. Cl. A61K 31/4184; G01N 33/573; A61K 31/4192; A61P 35/04; A61P 31/12; A61P 31/22; A61P 29/00

(52) U.S. Cl. 514/394; 435/7.4; 514/359

(57) ABSTRACT

Disclosed are methods related to label-free biosensor cellular assays to classify multienzyme complex modulators in live cells. Disclosed are also methods related to the identification of purinosome disrupting Casein Kinase II (CK2) inhibitors, and methods related to the use of purinosome disrupting CK2 inhibitors as therapeutic agents for modulating CK2 activity and purine synthesis pathway, and for improving prevention and treatment of CK2 associated cancers, viral infection and inflammation conditions.

### BACKGROUND

Label free biosensor assays are desirable assays for monitoring cell activities because they allow for non-invasive and real time cellular analysis. However, label free biosensor assays for many cellular activities have not been identified. Multienzyme complexes represent very interesting targets for the pharmaceutical industry, but because of their extreme complexity, desired assays do not exist. Disclosed are label free biosensor assays designed for studying multienzyme complexes such as purinosomes, and screening molecules regulating the dynamics of such multienzyme complexes. Also, disclosed are methods to prevent, treat, or cure cancerous diseases related to casein kinase II (CK2).

### SUMMARY OF THE INVENTION

The methods described herein are directed towards using label-free biosensor cellular assays for directly and indirectly detecting the cellular activity of multienzyme complexes, including purinosomes. The methods described herein are can be used with using label-free biosensor cellular assays for screening molecules, including CK2 inhibitors that regulate the dynamics of purinosome formation. The methods described herein are also to prevent, treat, or cure cancerous diseases related to the abnormal activity of CK2.

### BRIEF DESCRIPTION OF THE DRAWINGS

FIG. 1 shows the de novo purine synthesis pathway.

FIG. 2 shows the morphology and CK2 inhibitor-induced DMR signals of HeLa cells under different culture conditions. (A) Shows the light microscopy image of HeLa cells with initial seeding density of 5,000 cells per well; (B) Shows the light microscopy image of HeLa cells with initial seeding density of 10,000 cells per well; (C) Shows the light microscopy image of HeLa cells with initial seeding density of 20,000 cells per well; (D) Shows the CK2 inhibitor TBB-induced DMR signals of HeLa cells with three different seeding densities; and (E) Shows the CK2 inhibitor DMAT-induced DMR signals of HeLa cells with three different seeding densities. All cells were cultured on Epic® tissue culture compatible 384 well microplates for one day using regular serum-containing medium before assays. The TBB concentration was 100 μM, while the DMAT concentration was 25 μM. At least 8 replicates were used to generate each average response.

FIG. 3 shows CK2 inhibitor-induced DMR signals of HeLa cells cultured using purine depleted medium. (A) Shows the CK2 inhibitor TBB-induced DMR signals of HeLa cells; and (B) shows the CK2 inhibitor DMAT-induced DMR signals of HeLa cells. All cells were cultured on Epic® tissue culture compatible 384 well microplates using the purine depleted medium for one day before assaying. Two different seeding densities were used as indicated. The TBB concentration was 100 μM, while the DMAT concentration was 25 μM. At least 8 replicates were used to generate each average response.

FIG. 4 shows the dynamics of CK2 inhibitor-induced DMR signals of HeLa cells. (A) Shows that the CK2 inhibitor TBB induced DMR signal was reversed by the subsequent stimulation with DMAT. (B) Shows that the CK2 inhibitor DMAT induced DMR signal was also reversed by the subsequent stimulation with TBB. All cells were cultured on Epic® tissue culture compatible 384 well microplates for one day; and cells were cultured using regular serum medium with an initial seeding density of 25,000 cells per well. The TBB concentration was 50 μM and the DMAT concentration was 25 μM during all conditions. At least 4 replicates were used to generate the average responses.

FIG. 5 shows the CK2 inhibitor DMAT dose-dependent DMR signals of HeLa cells. (A) Shows real time responses; and (B) shows the amplitudes of DMR signals, 6 min and 50 min after DMAT stimulation, as a function of DMAT concentrations. Cells were cultured on Epic® tissue culture compatible 384 well microplates for one day using regular serum medium with an initial seeding density of 25,000 cells per well.

FIG. 6 shows the CK2 inhibitor TBB dose-dependent DMR signals of HeLa cells. (A) Shows real time responses; and (B) shows the amplitudes of DMR signals, at 50 min after TBB stimulation, as a function of TBB concentrations. Cells were cultured on Epic® tissue culture compatible 384 well microplates for one day using regular serum medium with an initial seeding density of 25,000 cells per well.

FIG. 7 shows the CK2 inhibitor dose-dependent DMR signals of HeLa cells. (A) Apigenin; (B) DRB; and (C) TBCA. Cells were cultured on Epic® tissue culture compatible 384 well microplates for two days using regular serum medium with an initial seeding density of 3,000 cells per well.

FIG. 8 shows that the dynamics of CK2 inhibitor-induced DMR signals of HeLa cells is sensitive to actin remodeling. (A) Shows the impact of actin disrupting agent latrunculin A on the dynamics of CK2 inhibitor-induced DMR signals in HeLa cells. The latrunculin A-pretreated cells were sequentially treated with TBB, and DMAT. (B) Shows the impact of actin promoting agent phalloidin on the dynamics of CK2 inhibitor-induced DMR signals in HeLa cells. The phalloidin-pretreated cells were sequentially treated with TBB, and DMAT. (C) Shows the impact of actin disrupting agent latrunculin A on the dynamics of CK2 inhibitor-induced DMR signals in HeLa cells. The latrunculin A-pretreated cells were sequentially treated with DMAT, and TBB. (D) Shows the impact of the actin promoting agent phalloidin on the dynamics of CK2 inhibitor-induced DMR signals in HeLa cells. The phalloidin pretreated cells were sequentially treated with DMAT, and TBB. Each stimulation step lasted about 1 hr. All cells were cultured on Epic® tissue culture compatible 384 well microplates for one day using regular serum medium with an initial seeding density of 25,000 cells per well. The TBB concentration was 50 μM and the DMAT concentration was 25 μM during all conditions; either actin remodeling agent was assayed at 10 μM. At least 4 replicates for each were used to generate the average responses. Only cellular responses upon stimulation with CK2 inhibitors were presented.

FIG. 9 shows that the dynamics of CK2 inhibitor-induced DMR signals of HeLa cells is sensitive to microtubule remodeling. (A) Shows the impact of microtubule remodeling agents on the dynamics of CK2 inhibitor inhibitor-induced DMR signals in HeLa cells. The cells were first pretreated with the microtubule remodeling agents, vinblastine or nocodazole, followed by treatment with TBB and DMAT. (B) Shows the impact of microtubule remodeling agents on the dynamics of CK2 inhibitor inhibitor-induced DMR signals in HeLa cells. The cells were first pretreated with the microtubule remodeling agents, vinblastine or nocodazole, followed by treatment with DMAT, and TBB. Nocodazole is a microtubule promoting agent and vinblastine is a microtubule disrupting agent. Each stimulation step lasted about 1 hr. All cells were cultured on Epic® tissue culture compatible 384 well microplates for one day using regular serum medium with an initial seeding density of 25,000 cells per well. Under all conditions, the TBB concentration was 50 μM, while the DMAT concentration was 25 μM, either microtubule remodeling agent was assayed at 10 μM. At least 4 replicates for each were used to generate the average responses. Only cellular responses upon stimulation with CK2 inhibitors were presented.

FIG. 10 shows the dynamics of CK2 inhibitor-induced DMR signals of HeLa cells was sensitive to endogenous Gi-coupled alpha2A-adrenergic receptor agonist oxymetazoline. (A) Shows the DMR signal of 10 μM oxymetazoline in HeLa cells. (B) Shows the impact of oxymetazoline pretreatment on the DMAT DMR signal in HeLa cells. (C) Shows the impact of oxymetazoline and DMAT pretreatment on the TBB DMR signal in HeLa cells. The cells were sequentially treated with oxymetazoline, DMAT, and TBB. Each step was monitored separately. Cells were cultured on Epic® tissue culture compatible 384 well microplates for one day using regular serum medium with an initial seeding density of 25,000 cells per well. The TBB concentration was 50 μM and the DMAT concentration was 25 μM during all conditions. At least 4 replicates for each were used to generate the average responses.

FIG. 11 shows that the dynamics of CK2 inhibitor-induced DMR signals of HeLa cells is insensitive to endogenous Gs-coupled beta2-adrenergic receptor agonist terbutaline. (A) Shows the DMR signal of 10 μM terbutaline in HeLa cells. (B) Shows the impact of terbutaline pretreatment on the DMAT DMR signal in HeLa cells. (C) Shows the impact of terbutaline and DMAT pretreatment on the TBB DMR signal in HeLa cells. The cells were sequentially treated with terbutaline, DMAT, and TBB. Each step was monitored separately. Cells were cultured on Epic® tissue culture compatible 384 well microplates for one day using regular serum medium with an initial seeding density of 25,000 cells per well. The TBB concentration was 50 μM and the DMAT concentration was 25 μM during all conditions. At least 4 replicates for each were used to generate the average responses.

FIG. 12 shows the impact of three Gi-coupled receptor agonists on the DMAT DMR signals in HeLa cells. (A) LPA (10 μM); (B) ACEA (10 μM); and (C) S1P (10 μM). The cells were pretreated with either agonist for about 1 hr. Only the DMAT response is shown. Cells were cultured on Epic® tissue culture compatible 384 well microplates for one day using regular serum medium with an initial seeding density of 25,000 cells per well. The DMAT concentration was 25 μM. At least 4 replicates for each were used to generate the average responses.

FIG. 13 shows the alpha2A-adrenergic receptor agonist clonidine dose-dependently potentiated the TBB DMR signal in HeLa cells. The cells were pretreated with clonidine for about 1 hr. Cells were cultured on Epic® tissue culture compatible 384 well microplates for one day using regular serum medium with an initial seeding density of 25,000 cells per well. The TBB concentration was 50 μM. At least 4 replicates for each were used to generate the average responses.

FIG. 14 shows the 10 μM TBB-mediated DMR signals in 6 different cell lines. (A) A431 cells; (B) A549 cells; (C) HEK293 cells; (D) MDA-AB-231 cells; (E) HT29 cells; and (F) PC3 cells. Before assaying the cells were cultured to be highly confluent on Epic® cell culture compatible 384 well microplates.

### DETAILED DESCRIPTION OF THE INVENTION

Disclosed are methods related to label-free biosensor cellular assays. Also disclosed herein are methods of screening and classifying CK2 modulators. The disclosed methods result in the discovery of distinct CK2 inhibitors that can regulate differently the dynamics (i.e., formation and dissociation) of purinosome complexes. The purinosome is a multi-enzyme signaling complex important for de novo purine synthesis pathway. The disclosed methods utilize CK2 inhibitors (in example TBB and DMAT) to track the association (i.e., formation) and dissociation of purinosomes in real time in native cells. The disclosed methods are preferably use pre-synchronized cells. The cells can be pre-synchronized to a specific state such that the CK2 inhibitor induced dynamics of purinosomes can be robustly measured using label-free biosensor cellular assays.

Also disclosed herein are methods of studying upstream signaling pathways that are involved in the purinosome formation. The disclosed methods can use the CK2 inhibitor induced DMR signal in native cells as a readout to identify its upstream pathways.

Also disclosed herein are methods of classifying CK2 inhibitors into purinosome-promoting and purinosome-disrupting CK2 inhibitors. The disclosed methods in certain embodiments are based on the similarity of a molecule primary DMR signal to a known CK2 inhibitor DMR in the same cell, or the modulation pattern of a molecule against the two different CK2 inhibitor TBB and DMAT DMR signals, or the similarity of a molecule modulation index to a known CK2 inhibitor acting on panels of cells/markers.

Also disclosed herein are methods of preventing, treating and curing cancerous diseases related to the abnormal activity of CK2. The disclosed methods use purinosome-disrupting CK2 inhibitors as anti-cancer therapeutic agents. Since purinosome formation is important for de novo purine synthesis, and inhibition of the purine synthesis pathway has been proven to be a viable approach for anti-cancer therapy, purinosome-disrupting CK2 inhibitors can be used as anti-cancer therapy agents for preventing, treating and curing the cancerous diseases related to the abnormal activity of CK2.

Disclosed are a variety of classes of compositions, compounds, methods, and method steps, and these classes and specific examples of each can be used, for example, in the methods disclosed herein. Disclosed are cell systems and their use for the identification of, for example, CK2 inhibitors, or multienzyme complex promoting agents or disrupting agents. The cell systems can be incubated with any of the agents or molecules, for example, disclosed herein, which form incubated cell systems. The cell systems can be analyzed and assayed with label free biosensor systems as disclosed herein. In certain systems, the cell systems can include synchronized cells. The cell systems, or any other cells, can be cultured in purine rich serum mediums. In certain embodiments the cell systems and cells disclosed herein can be cultured from a high initial seeding number and/or to a high confluency. The cell systems and cells disclosed herein can also be cultured in purine depleted mediums and/or under long term starvation conditions to a highly confluent cell population. The cell systems can be a transformed cell line, an immortalized cell line, a primary cell, and a stem cell.

Disclosed are multienzyme complexes which can be manipulated using the reagents and methods disclosed herein. Also disclosed are multienzyme complex modulators, multienzyme complex promoting agents, multienzyme complex disassembly promoting agents, assembly promoting agents, disassembly promoting agents, purinosome complex disrupting agents, purinsome inhibitor agents, purinosome complex promoting agents, purinosome promoting agents, purinosome-dissembling CK2 inhibitors, purinsome disrupting CK2 inhibitors, purine synthesis pathway inhibitors, CK2 modulators, CK2 activators, CK2 inhibitors, and reference probes all of which can be used with any of the reagents and compositions and compounds disclosed herein, along with the methods disclosed herein.

A variety of molecules having activities disclosed herein are also disclosed and can be used in any combination or alone in the methods discussed herein, such as TBB (4,5,6,7-tetrabromobenzotriazole), DMAT (2-Dimethylamino-4,5,6,7-tetrabromo-1H-benzimidazole), and TBBz (4,5,6,7-tetrabromobenzimidazole).

Also disclosed are a variety of activities and cellular responses which can be assayed, such as CK2 activity, molecule induced biosensor responses, and reference probe biosensor responses. These responses can be assayed in a variety of cells including the cell systems disclosed herein, as well as native cells and CK2 inhibitor sensitive cells. In certain embodiments, the preactivation of endogenous Gi-coupled receptors can be assayed, and their modulation and impacts on the CK2 inhibitor induced biosensor cellular responses in the same cell can be monitored.

The assays can be used to monitor the association and dissociation of purinosomes in real time.

The compositions and compounds identified by the disclosed methods can be used in the treatment of cancers, such as leukemia, colorectal cancer, prostate cancer, breast cancer, and lymphoma, squamous cell carcinoma of head and neck, lung, brain glioma cancer or a cancer phenotype arising from the genetic alteration of onco-kinases, such as Abl or Alk. Likewise the identified compositions and compounds can be used to treat viral infections, such as those caused by Herpes virus or Cytomegalovirus and inflammation conditions, such as chronic inflammation disorder or condition, such as intestinal disorders, such as inflammatory bowel disease, ulcerative colitis, and Crohn's disease can be a chronic inflammation disorder or condition and glomerulonephritis is also a chronic inflammation disorder. These diseases are often associated with the abnormal activity of CK2 kinases.

In all cases the compositions and compounds disclosed herein can be administered in therapeutically effective amounts.

The methods disclosed herein, as well as the compositions and compounds which can be used in the methods, can arise from a number of different classes, such as materials, substance, molecules, and ligands. Also disclosed is a specific subset of these classes, unique to label free biosensor assays, called markers, for example, mallotoxin as a marker for hERG activation, and DMAT as a marker for purinosome association.

It is understood that mixtures of these classes, such as a molecule mixture are also disclosed and can be used in the disclosed methods.

In certain methods, unknown molecules, test molecules, drug candidate molecules as well as known molecules can be used.

In certain methods or situations, modulating or modulators play a role. Likewise, known modulators can be used.

In certain methods, as well as compositions, cells are involved, and cells can undergo culturing and cell cultures can be used as discussed herein.

The methods disclosed herein involve assays that use biosensors. In certain assays, they are performed in either an agonism or antagonism mode. Often the assays involve treating cells with one or more classes, such as a material, a substance, or a molecule. It is also understood that subjects can be treated as well, as discuss herein.

In certain methods, contacting between a molecule, for example, and a cell can take place. In the disclosed methods, responses, such as cellular response, which can manifest as a biosensor response, such as a DMR response, can be detected. These and other responses can be assayed. In certain methods the signals from a biosensor can be robust biosensor signals or robust DMR signals.

The disclosed methods utilizing label free biosensors can produce profiles, such as primary profiles, secondary profiles, and modulation profiles. These profiles and others can be used for making determinations about molecules, for example, and can be used with any of the classes discussed herein.

Also disclosed are libraries and panels of compounds or compositions, such as molecules, cells, materials, or substances disclosed herein. Also disclosed are specific panels, such as marker panels and cell panels.

The disclosed methods can utilize a variety of aspects, such as biosensor signals, DMR signals, normalizing, controls, positive controls, modulation comparisons, Indexes, Biosensor Indexes, DMR indexes, Molecule biosensor indexes, molecule DMR indexes, molecule indexes, modulator biosensor indexes, modulator DMR indexes, molecule modulation indexes, known modulator biosensor indexes, known modulator DMR indexes, marker biosensor indexes, marker DMR indexes, modulating the biosensor signal of a marker, modulating the DMR signal, potentiating, and similarity of indexes.

Any of the compositions, compounds, or anything else disclosed herein can be characterized in any way disclosed herein.

Disclosed are methods that rely on characterizations, such as higher and inhibit and like words.

In certain methods, receptors or cellular targets are used. Certain methods can provide information about signaling pathway(s) as well as molecule-treated cells and other cellular processes.

In certain embodiments, a certain potency or efficacy becomes a characteristic, and the direct action (of a drug candidate molecule, for example) can be assayed.

The disclosed methods can be performed on or with samples.

### A. COMPOSITIONS

1. Casein Kinase II or Protein Kinase CK2 (CK2)

Casein Kinase II or protein kinase CK2 (CK2) is a constitutively active serine/threonine protein kinase composed of two 44 kDa catalytic α-subunits and two 26 kDa regulatory β-subunits in an α2β2 configuration to form stable heterotetramers. Two catalytic subunits (α and α′) and two regulatory subunits (β2) in three configurations (α2β2, αα′β2 or α′2β2) constitute the heterotetrameric CK2 holoenzymes. However, individual catalytic subunits (CK2α or CK2α′) can also function without regulatory subunits. CK2 holoenzyme undergoes autophosphorylation at two serine residues (S2/S3) of its β-subunit. Also, CK2 is able to phosphorylate, under special circumstances, for example, on tyrosyl residues in proteins. The CK2β subunit exhibits several regulatory functions related to the stability, activity and specificity of CK2α and CK2α′ in the holoenzyme complex. In addition, the three-dimensional structure of the CK2β dimer and the identification of several important protein kinases that interact with CK2β in the absence of CK2 catalytic subunits strongly indicates that CK2β has functions distinct from CK2α.

CK2 is a pleiotropic, multifaceted, conserved, ubiquitous protein kinase involved in a large number of cellular processes, including cell growth, proliferation and suppression of apoptosis. CK2 is capable of dynamic intracellular shuttling in response to a variety of signals. In normal cells, CK2 is localized in both the nucleus and cytoplasm, but in cancer cells CK2 is particularly predominant in the nuclear compartment, resulting in abnormal activity.

CK2 is essential to cell survival because it counteracts ‘premature’ apoptosis whenever it can be avoided without compromising ‘healthy’ viable cells. However, abnormally high CK2 activity can also prevent programmed cell death where this is timely and appropriate. Thus, for example, CK2 can enhance the neoplasia tumor phenotype, whose key feature is deregulation of apoptosis. Downregulation of CK2 by chemical or molecular methods can promote apoptosis in cells.

2. CK2 and Cancers

CK2 has been found to be uniformly dysregulated in most tested cancers. Aberrant activation of protein kinases is a key oncogenic force underlying human tumorigenesis. Traditionally, CK2 has been regarded as a constitutively active protein-kinase in search of specific cellular functions. However, several studies indicate that CK2 is a stress-activated kinase that plays a crucial role in the regulation of cell proliferation and in the transduction of survival signals. Recently, several molecular pathways modulating the prosurvival properties of CK2 have started to emerge: (1) CK2 is activated by UV radiation in a p38 MAPK-dependent manner, leading to the phosphorylation and degradation of the NFκB inhibitor IκBa. Upon UV irradiation, CK2 also complexes and phosphorylates p53 at Ser389. Conversely, wild-type p53 inhibits CK2 activity, supporting the notion that p53 and CK2 are interconnected in a tightly regulated network. (2) CK2 is frequently activated in human cancers and can induce mammary tumors and lymphomas when expressed in transgenic mice. (3) Altered CK2 activity in human cancers leads to phosphorylation and degradation of the tumor-suppressor protein PML through integration of upstream p53 and p38MAPK signals. (4) Hypoxia-induced activation of HIF-1 in cancer cells is mediated by a CK2-dependent down-regulation of p53. (5) A moderate down-regulation of nuclear-associated CK2α leads to growth arrest and induction of apoptosis in prostate cancer cells and CK2α nuclear localization is associated with poor prognostic factors in human prostate cancer. Thus, CK2 is related to cancer and is considered a suitable target for cancer therapy.

Importantly, it has been reported that antisense RNA-mediated CK2α down-regulation induces potent apoptosis in cancer cells, but minimal cell death in normal cells. These observations indicate that CK2 should have disease-associated functions which are separate from its normal functions. This raises the possibility of a pharmacological window in targeting CK2 for induction of apoptosis in cancer cells under conditions that can spare normal cells. Given the subcellular dynamic of CK2 subunits and the transient nature of their interactions in living cells, the use of potent and specific inhibitors is the first-choice approach for manipulating this kinase. However, the development of such molecules has remained limited in the absence of useful in vivo screening models for molecules capable of modulating CK2 activity. In addition, the few molecules described, capable of inhibiting CK2 activity, including ATP analogs such as TBB, IQA and condensed polyphenolic derivatives, have the drawback of having poor specificity and/or poor activity.

3. CK2 Inhibitors

CK2 inhibitors can provide anti-cancer and anti-inflammatory potential. CK2 inhibitors are commonly classified into three categories: (1) inhibitors that target the regulatory subunit of CK2 (e.g., genetically selected peptide aptamers); (2) inhibitors of the catalytic activity of CK2 (e.g., quinobene, TBB, DMAT, IQA); and (3) disruptors of CK2 holoenzymes, which are often molecules binding to the CK2 subunit interface and inhibit the high affinity interaction of its subunits. The CK2 inhibitors of each class can be any type of molecule, such as a, small molecules, functional nucleic acids, antibodies, or peptide mimetics, etc.

CK2 catalytic subunits possess a constitutive activity. However, in eukaryotic cells the CK2β subunits are not only the central components of the tetrameric CK2 complex, but are also responsible for the recruitment of CK2 substrates. Thus, the dynamic interaction of the CK2 subunits observed in living cells can have a key role in CK2 signaling pathways. Drugs that specifically target this interaction are less likely to have side effects than drugs that act as general inhibitors of CK2 catalytic activity.

CK2 inhibitors consist of a diverse array of chemicals, including flavonoids (e.g. apigenin), derivatives of hydroxyantraquinones/xantenones (e.g., emodin), derivatives of hydroxycoumarines (e.g., DBC), derivatives of tetrabromotriazole/imidazole (e.g., DRB, TBB, DMAT, TBCA, TBBz), and derivatives of indoloquinazolines (e.g., IQA).

A number of structurally unrelated CK2 inhibitors display a pro-apoptotic effect (approximately proportional to their in vitro inhibitory potency) when tested on a variety of cells derived from tumors, including lymphomas, leukaemias, multiple myeloma and prostate carcinoma. The incontrovertible evidence that such a cytotoxic effect is really due to CK2 inhibition has been provided with HEK-293 cells (human embryonic kidney cells) by showing that apoptosis induced by the CK2 inhibitor K27 is abrogated if the cells are transfected with the CK2 V66A/I174A mutant, which is 11-fold less sensitive to K27 than wild-type CK2.

It is known that CK2 can counteract programmed cell death via several different mechanisms, including acceleration of IkB (inhibitory kB) degradation by calpain, generation of cleavage-resistant sites in a variety of caspase protein substrates, activation of the caspase inhibitor protein ARC (apoptosis repressor with caspase recruitment domain), potentiation of the Akt/PKB (protein kinase B) pathway and facilitation of DNA repair.

The usage of cell permeable CK2 inhibitors with reduced or absent undesirable side effects provides a new strategy to combat tumors as well as infections by viruses, which are known to exploit the CK2 activity of the host cell to phosphorylate proteins essential to their life cycle. In particular, whenever a therapeutic strategy is based on induction of apoptosis (e.g. chemotherapy and radiotherapy), it is expectedly hampered by elevated CK2 activity and therefore CK2 inhibitors can display an adjuvant effect.

4. Metabolic Disorders Related to Purine Pathways

A metabolic disorder refers to distress in the body resulting from overproduction of toxic substance or underproduction of an essential substance. Metabolic diseases are inherited and present from birth, although the disease can manifest itself at any age. The most familiar metabolic diseases are: Sickle Cell Anemia (caused by a defect in the protein which carries oxygen), Cystic Fibrosis (resulting from a defect in the enzyme that transports salt) and Lactose Intolerance (caused by a defect in the enzyme which digests the sugar (lactose) in milk). Considering the many t roles purines play in human metabolism, it is not surprising that the diseases of purine metabolism are as varied, ranging from asymptomatic conditions, which are only discovered accidentally, to disorders with severe neurological abnormalities, which are ultimately fatal. As with other metabolic diseases, each disorder is caused by a defective gene which results in an enzyme with too little or too much catalytic activity. Purine metabolic diseases include:

i. Gout

Gout is caused by overproduction of uric acid, with a consequent depositing of uric acid crystals in the joints. Several different enzyme defects cause gout, notably deficiency of HPRT. Gout can be treated successfully by limiting purines in the diet and by using drugs which inhibit xanthine oxidase and, thereby, the production of uric acid.

ii. Lesch-Nyhan Syndrome

Lesch-Nyhan Syndrome is caused by a deficiency of HPRT. Symptoms include very severe gout, poor muscular control (patients are wheelchair-bound), and moderate mental retardation.

iii. Adenosine Deaminase (ADA) and Purine Nucleoside Phosphorylase (PNP) Deficiency.

A deficiency of either ADA or PNP causes a moderate to complete lack of immune function.

iv. Adenylosuccinate Lyase Deficiency

A deficiency of adenylosuccinate lyase enzymes results in mental retardation, seizures, and autistic behavior.

v. Myoadenylate Deaminase Deficiency

A deficiency of myoadenylate deaminase impairs the ability of muscles to regulate energy during exercise. The most prominent symptoms are muscle fatigue and cramps after normal activities, such as climbing stairs. Several experimental therapies appear to be helpful.

vi. 5′ Nucleotidase Defect

The most recently described and most unusual defect of purine metabolism is caused by excessive activity of the enzyme 5′ nucleotidase. Symptoms include constant infections, seizures, skin rashes, and very unusual behavior, characterized by extreme hyperactivity, short attention span, lack of speech, and poor social interaction. This disease appears to be fully treatable by diets which restore the compounds that are consumed by the excessive enzyme activity.

vii. Phosphoribosyl Pyrophosphate (PRPP) Synthetase Defects

Two distinct defects are associated with PRPP synthetase. Enzyme deficiency results in convulsions, autistic behavior, anemia, and severe mental retardation. Excessive enzyme activity causes gout, along with various neurological symptoms, such as deafness. Aside from the treatment of gout, no treatment for the symptoms of these diseases is available at this time.

viii. Xanthinuria and Adenine Phosphoribosyltransferase (APRT) Deficiency.

A deficiency of either xanthine oxidase or APRT causes accumulation of xanthine or 2,8 dihydroxyadenine, respectively. APRT often causes no symptoms at all, and patients are discovered accidentally during some other kind of medical test. In other cases, these compounds accumulate and crystallize in the joints, causing a gout-like condition. No effective treatment is known, though reduction in dietary purines is often helpful.

5. Purinosomes, CK2, and Metabolic Pathways

i. Purinosomes

Proteins are likely to organize into complexes that assemble and disassemble depending on cellular needs. In a quiescent state, a large number of proteins involved in intermediary metabolism and stress response were observed to form punctate cytoplasmic foci. The purine biosynthetic enzyme Ade4-GFP formed foci in the absence of adenine, and cycling between punctate and diffuse phenotypes could be controlled by adenine subtraction and addition. Similarly, glutamine synthetase (Gln1-GFP) foci cycled reversibly in the absence and presence of glucose. The structures were neither targeted for vacuolar or autophagosome degradation nor colocalized with P bodies or major organelles. Thus, upon nutrient depletion, the cell induces widespread protein assemblies displaying nutrient-specific formation and dissolution.

A recent study using fluorescence microscopy to HeLa cells (An, S. et al. “Reversible compartmentalization of de novo purine biosynthetic complexes in living cells”. Science 2008, 320: 103-106) indicates that all six enzymes related to purine synthesis pathway colocalize to form clusters in the cellular cytoplasm. The association and dissociation of these enzyme clusters can be regulated dynamically, by either changing the purine levels of or adding exogenous agents to the culture media. This finding provides strong evidence for the formation of a multi-enzyme complex, the “purinosome,” to carry out de novo purine biosynthesis in cells.

CK2 and Akt (also known as protein kinase B) have been implicated to interact with de novo purine biosynthetic enzymes based on two different in vitro proteomic scale experiments; i) hPPAT, hTrifGART and hFGAMS are substrates for hCK2, and ii) hFGAMS is a substrate for Akt. Several key metabolic enzymes were described to be implicated as substrates of CK2 or Aid; for example, glycogen synthase, acetyl-CoA carboxylase and ornithine decarboxylase for CK2, and ATP-citrate lyase for Akt.

ii. Metabolic Pathways and System

Coordinated and highly regulated metabolic processes are essential to biological function and occur in all components of the human body. The human body extracts hydrocarbons from ingested food and transforms the potential chemical energy in these nutrients to ATP, which ultimately fuels all physiological processes.

Each metabolic pathway is composed of a series of biochemical reactions that are connected by their intermediates: The reactants (or substrates) of one reaction are the products of the previous one, and so on. Metabolic pathways are usually considered in one direction (although all reactions are chemically reversible, thermodynamical conditions in the cell are favorable for flux to be in one of the directions). Glycolysis was the first discovered metabolic pathway. As glucose enters a cell, it is immediately irreversibly phosphorylated by ATP to glucose 6-phosphate. This prevents glucose from leaving the cell. In times of excess lipid or protein energy sources, glycolysis can run in reverse (gluconeogenesis) to produce glucose 6-phosphate for storage as glycogen or starch.

Metabolic pathways are often regulated by feedback inhibition, or by a cycle wherein one of the products in the cycle restarts the reaction, such as the Krebs Cycle.

Anabolic and catabolic pathways in eukaryotes are separated either by compartmentalization or by the use of different enzymes and cofactors. Several distinct, but linked, metabolic pathways are used by cells to transfer the energy released by breakdown of fuel to ATP: glycolysis, anaerobic respiration, Krebs cycle/Citric acid cycle and oxidative phosphorylation. Other pathways occurring in (most or) all living organisms include: Fatty acid oxidation (β-oxidation), Gluconeogenesis, HMG-CoA reductase pathway, Pentose phosphate pathway (hexose monophosphate shunt), Porphyrin synthesis (or heme synthesis) pathway, and Urea cycle.

iii. Purine Synthesis and the Purinosome

Purines play many important biological roles for life to exist. Different metabolic pathways exist for: (1) making purines (the synthetic pathways); (2) converting purine compounds (the conversion pathways); (3) reusing purines consumed in the diet (the reuse pathways); and (4) disposing of excess purines (the disposal pathways). Similarly to information molecules in genes, purines are used in the process of converting genes to proteins. As energy transducers in cellular signaling processes, such as nerve conduction and muscle contraction, they act as messengers. As a disposal mechanism, they remove excess nitrogen from cells. As antioxidants, they protect the cell from cancer-causing agents.

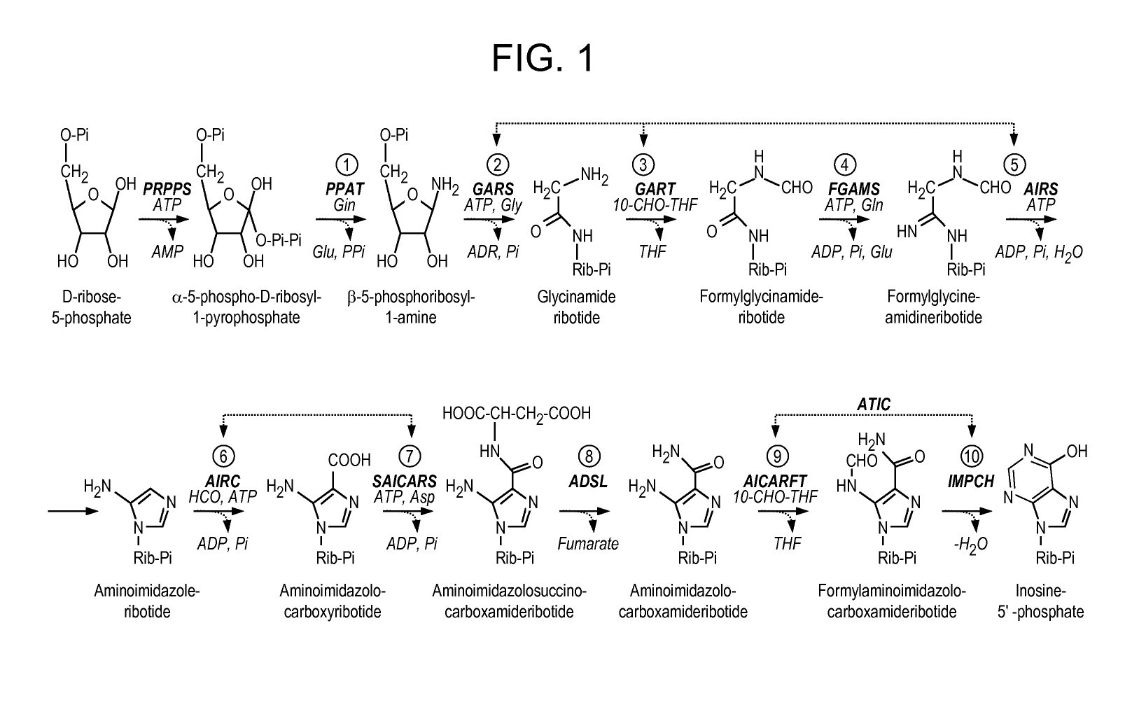

Purines are not only essential building blocks of DNA and RNA, but also of nucleotide derivatives were they participate in a multitude of pathways in both prokaryotes and eukaryotes. Biosynthetically, adenosine and guanosine nucleotides are derived from inosine monophosphate (IMP), which is synthesized from phosphoribosyl pyrophosphate (PRPP) in both the de novo and salvage biosynthetic pathways. The salvage pathway catalyzes the one-step conversion of hypoxanthine to IMP by hypoxanthine phosphoribosyl transferase (HPRT), whereas the de novo pathway consists of 10 chemical reactions that transform PRPP to IMP (see FIG. 1). In higher eukaryotes (such as humans), the de novo pathway uses six enzymes, including three multifunctional enzymes: a trifunctional protein, TrifGART (that has glycinamide ribonucleotide (GAR) synthetase (GARS), GAR transformylase (GAR Tfase), and aminoimidazole ribonucleotide synthetase (AIRS) activities); a bifunctional enzyme, PAICS, (that has carboxyaminoimidazole ribonucleotide synthase (CAIRS) and succinylaminoimidazolecarboxamide ribonucleotide synthetase (SAICARS) activities); and a bifunctional enzyme, ATIC, (that has aminoimidazolecarboxamide ribonucleotide transformylase (AICAR Tfase) and IMP cyclohydrolase (IMPCH) activities). In contrast, prokaryotes, such as Escherichia coli, use only monofunctional enzymes throughout this pathway, except for the bifunctional ATIC. These enzymes can form a multienzyme complex, depending on cellular status. The association and dissociation of the multienzyme complex can be regulated by cellular purine levels that were imposed by the addition of external reagents that regulate purine metabolic flux. These functional complexes can produce efficient substrate channels that link the 10 catalytic active sites. Additionally, clustering of the 10 active sites can provide efficient means of globally regulating purine flux under varying environmental conditions. These multienzyme complexes observed in the de novo purine biosynthetic pathway can constitute a “purinosome.” The formation of the purinosome is dynamically regulated by stimulation of de novo purine biosynthesis in response to changes in purine levels. The purinosome can be a general phenomenon in all cell types during specific stages of the cell cycle, along with posttranslation modifications. Because of the relevance of de novo purine biosynthesis to human diseases, the purinosome can represent a new pharmacological opportunity for therapeutic intervention.

6. Purine Pathway Inhibitors as Anti-Cancer Agents

Inhibition of cellular replication is one characteristic of cancer cells that has been effectively exploited in the past for the development of anticancer agents. Most drugs that kill cancer cells inhibit the synthesis of DNA or interfere with its function in some way. For a cell to divide into two cells, it must replicate all components including its genome, and unlike the synthesis of other major macromolecules (protein, RNA, lipid, etc.), the synthesis of DNA does not occur to a great degree in quiescent cells. In an adult organism most cells are quiescent and are not in the process of duplicating their genome, therefore, drugs targeting DNA replication affords some level of selectivity. Of course, certain tissues (bone marrow, gastrointestinal, hair follicles, etc.) are in a replicative state, and all cells must continually repair their DNA. Therefore, inhibition of DNA replication in normal tissues results in considerable toxicity which limits the amount of drug that can be tolerated by the patient. However, very effective anticancer drugs have been developed that increase survival and, in some cases, cure the patient of his or her disease. Human cells have the capacity to salvage purines and pyrimidines for the synthesis of deoxyribonucleotides that are used for DNA synthesis, and analogues of these nucleotide precursors have proven to be an important class of anticancer agents. There are 14 purine and pyrimidine antimetabolites that are approved by the FDA for the treatment of cancers, which account for nearly 20% of all cancer drugs. Some of the first cancer drugs FDA approved were in this class of compounds. 6-Mercaptopurine was approved in 1953 for the treatment of childhood leukemia, where it is curative and is still the standard of treatment for this disease. Since 1991, nine nucleoside analogues were approved by the FDA for the treatment of various malignancies. Four of these new agents were approved since 2004, and there are numerous agents that are currently being evaluated in clinical trials. The recent FDA approvals indicate that the design and synthesis of new nucleoside analogues is still a productive area for discovering new drugs for the treatment of cancer. In general, these compounds have been most useful in the treatment of hematologic malignancies, and even though there is still room for significant improvements in the treatment of these diseases, some of the newer agents are finding use in the treatment of solid tumors.

The basic mechanism of action of purine and pyrimidine antimetabolites is similar. These compounds diffuse into cells (usually with the aid of a membrane transporter) and are converted to analogues of cellular nucleotides by enzymes of the purine or pyrimidine metabolic pathway. These metabolites then inhibit one or more enzymes that are critical for DNA synthesis, causing DNA damage and induction of apoptosis. Even though the compounds in this class are structurally similar and share many mechanistic details, it is clear that subtle quantitative and qualitative differences in the metabolism of these agents and their interactions with target enzymes can have a profound impact on their antitumor activity.

Potent inhibitors of purine (as well as pyrimidine) nucleotide biosynthesis can be either synthetic or natural-product analogues of intermediates of the pathway or, inhibitors can also be designed based of the catalytic mechanism. These inhibitors are effective drugs against cancer, inflammatory disorders and various infections. For treatment of human cancer, targeting the purine pathway is more common than targeting the pyrimidine pathway, where more toxic side effects are apparent. Drugs such as methotrexate have multiple sites of action, making it difficult to quantitatively predict their effects on cells. Design of inhibitors based on the X-ray structure of the target enzyme can yield drugs with only one site of action in human cells. Such approach resulted in the discovery of drugs acting against PPAT (e. g. piritrexm), GART (e.g., azaserine, diazomycin, dideazatetrahydrofolate, lometrexol), AIRC (fluorosulfonylbenzoyl-adenosine) and SAICARS (e.g, nitroaminoimidazole ribonucleotide). Purine de nova synthesis (PDNS) inhibitors cause disappearance of intracellular nucleotides and accumulation of PDNS intermediates to a mM concentration level.

7. Nucleic Acid Related Molecules

i. Nucleic Acids

There are a variety of molecules disclosed herein that are nucleic acid based, including for example, nucleic acids that function as modulators of, for example purinosomes, multienzyme complexes, and CK2 complexes. These can be termed functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989,86, 6553-6556).

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b. Sequences

There are a variety of sequences related to, for example, disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

c. Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

d. Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with, for example, purinosomes, multienzyme complexes, and CK2 complexes. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹² M. A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly to the target molecule with k_{d}s of less than 10⁻¹² M. It is preferred that the aptamers bind the target molecule with a k_{d} less than 10⁻⁶, 10⁻⁸, 10⁻¹⁰, or 10⁻12 M. Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of aptamers, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹²M. Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

8. Antibodies

i. Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with the compositions and compounds disclosed herein, such as purinosomes, multienzyme complexes, and CK2 complexes. Antibodies that bind specific regions of purinosomes, multienzyme complexes, and CK2 complexes are also disclosed. The antibodies can be tested for their desired activity using the in vitro and ex vivo assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

a. Human antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

b. Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5, 939,598 (Kucherlapati et al.), U.S. Pat. No. 6,130,364 (Jakobovits et al.), and U.S. Pat. No. 6,180,377 (Morgan et al.).

c. Administration of Antibodies

Administration of the antibodies can be done as disclosed herein. Nucleic acid approaches for antibody delivery also exist. The broadly neutralizing antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### B. METHODS

Disclosed herein are methods to re-classify CK2 inhibitors. The methods provide means for measuring the ability of CK2 inhibitors to modulate (either promote or disassemble) the dynamic multi-enzyme complexes that are important for purine synthesis. Some aspects of the methods use label-free cellular pharmacology based approach and are capable of identifying CK2 inhibitors that act as purine synthesis pathway inhibitors. Inhibition of purine synthesis pathways has been proven to be viable anti-cancer therapeutic strategy.

Disclosed are methods related to the use of purinosome-dissembling CK2 inhibitors as anti-cancer therapy agents, which can be used for preventing, treating or curing cancerous diseases or inflammatory disorders related to abnormal activity of CK2.

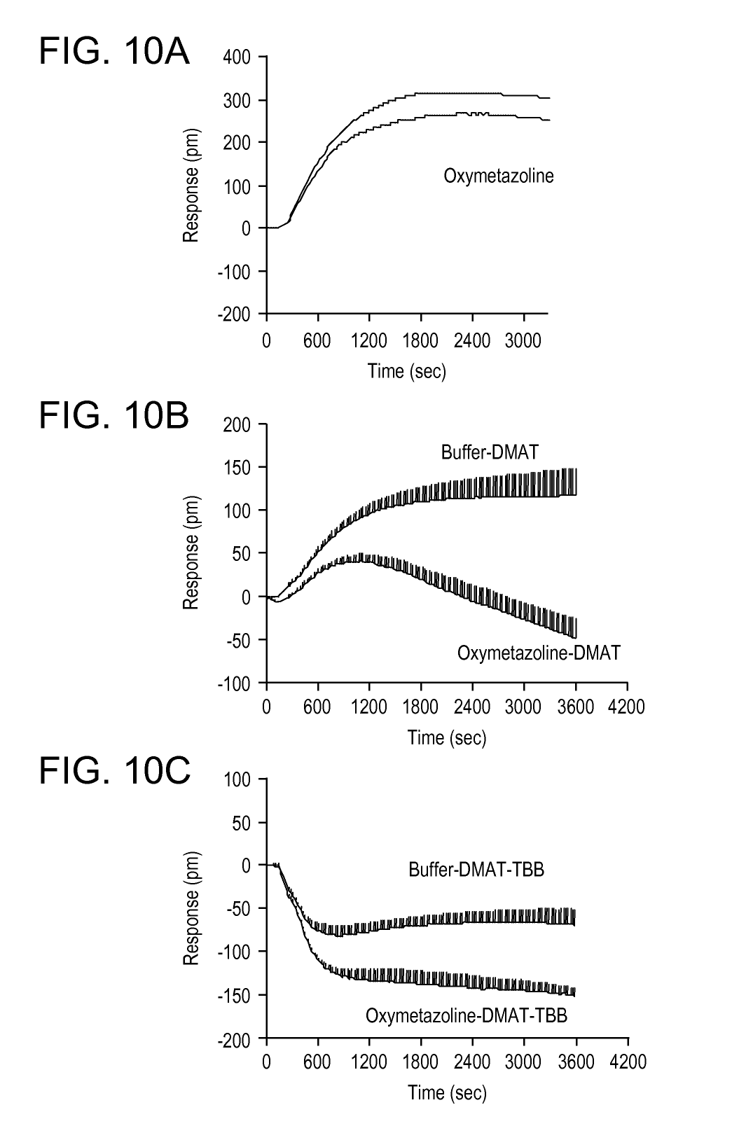

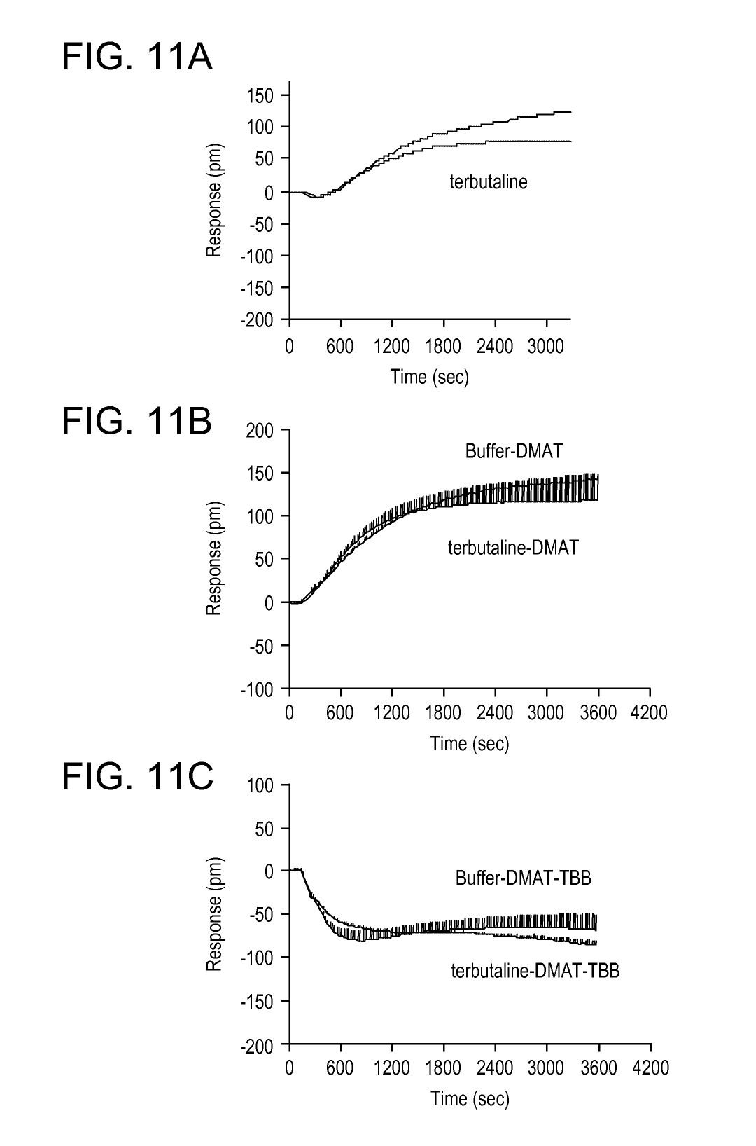

The present invention discloses methods to screen, and more specifically, classifying CK2 modulators. Disclosed are methods related to label-free biosensor cellular assays. Disclosed are methods related to the use of label-free biosensors for detecting CK2 activity in live cells and screening CK2 inhibitors using synchronized cells. Disclosed are methods related to the use of a pair of CK2 inhibitors, TBB and DMAT, as referencing probe to classify CK2 inhibitors in terms of their ability to either promoting or dissembling the purinosome complexes. The classification can be achieved by (1) comparing a molecule-induced DMR signal with the referencing probe-induced DMR signals wherein the similarity in DMR signals is an indicator that the molecule is a CK2 inhibitor or not; (2) studying the impacts of a molecule on both DMAT and TBB-induced DMR signals wherein the molecule that selectively inhibits the TBB DMR signal but potentiates the DMAT DMR signal, or vice versa, is a CK2 inhibitor; and (3) comparing molecule-induced DMR signals across a panel of CK2 inhibitor sensitive cells wherein the molecule that is active and behaves similar to known CK2 inhibitors in almost all cells, such as at least 70%, 80%, 90%, 95% is a CK2 inhibitor. Additionally, the sensitivity of a molecule induced DMR signal to the preactivation of endogenous Gi-coupled receptors can be used for further confirmation that the molecule is a CK2 inhibitor. Any combinations of the above methods can be used to further confirm the molecule being a CK2 inhibitor, and to separate a CK2 inhibitor from an upstream pathway modulator. For example, an agonist for endogenous Gi coupled receptor can inhibit the DMAT DMR, but potentiate the TBB DMR signal. However, the agonist itself will result in a distinct DMR signal in the same cell that is different from either TBB or DMAT DMR signals (see FIG. 10, as example); therefore, the agonist is a purinosome formation upstream modulator. On the other hand, an agonist for endogenous Gs coupled receptor can result in a DMR similar to the DMAT DMR, but has little effect on both DMAT and TBB DMR signals; therefore, the agonist is not a CK2 inhibitor (see example in FIG. 11).

The cells are preferably synchronized. The cell synchronization can be achieved by (1) culturing cells in serum and purine rich medium with a high initial seeding number such that the cells once attached reach high confluency; (2) culturing cells in serum but purine depleted medium; and (3) long term starvation of a highly confluent cell. Cell attachment is a rapid process, and particularly takes place within the first several hours (−0.5-8 hrs). Such cell pre-synchronization to a specific state is important for robustly detecting the CK2 inhibitor induced dynamics of purinosomes using label-free biosensor cellular assays.

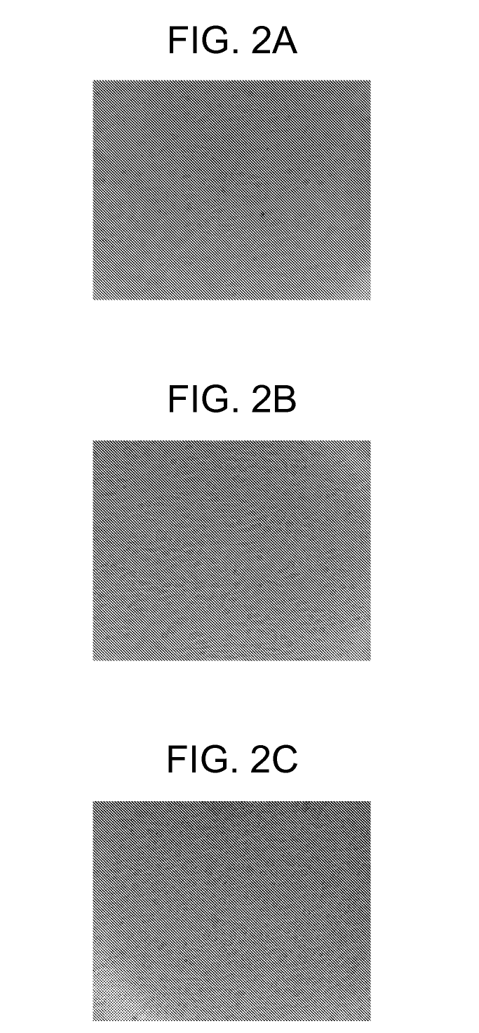

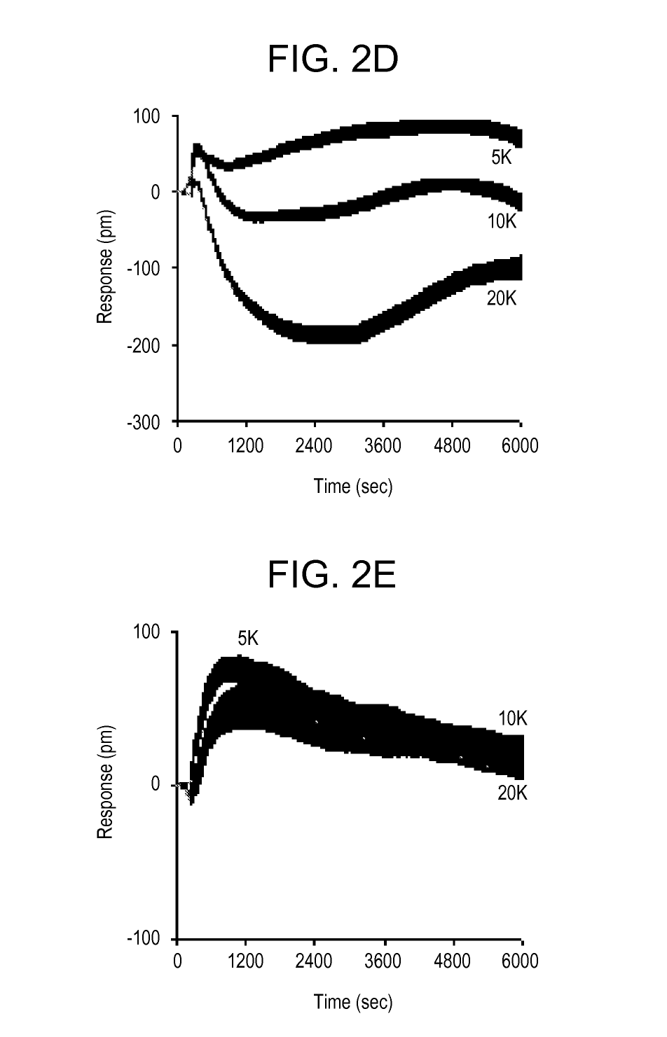

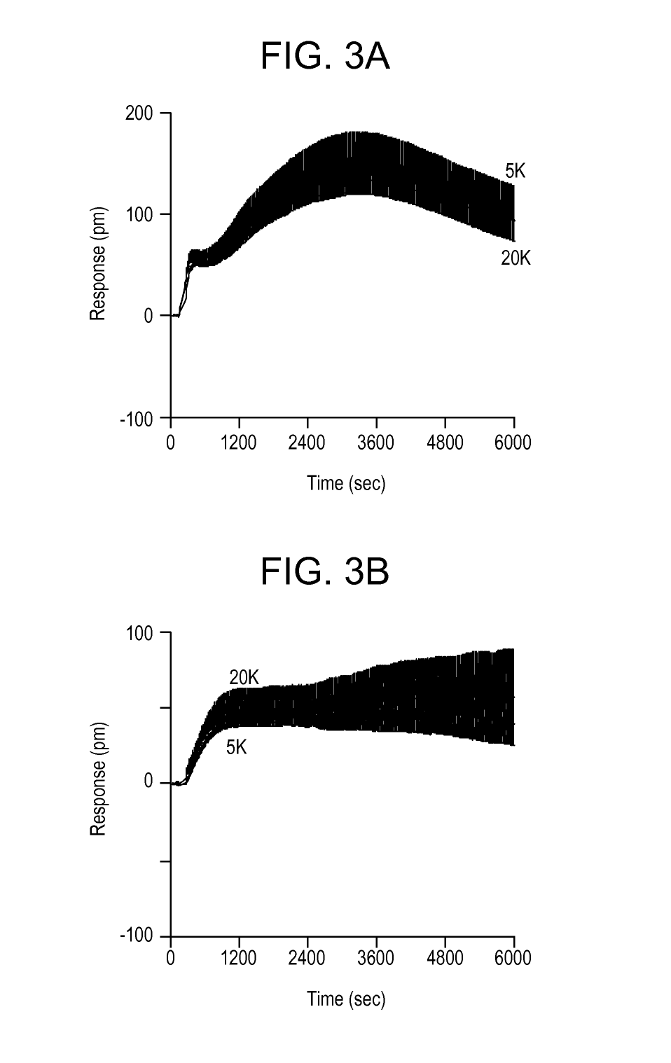

Also, disclosed are methods to classify CK2 inhibitors into purinosome-promoting and purinosome-disrupting CK2 inhibitors. The disclosed methods take advantage of the regulatory roles of CK2 in the dynamics of purinosome, a multi-enzyme signaling complex important for de novo purine synthesis pathway, and utilize a pair of CK2 inhibitors (TBB and DMAT) to track the association and dissociation of purinosomes in real time in native cells. According to the present invention, TBB is a purinosome complex disrupting agent, while DMAT is a purinosome promoting agent. However, it is worthy noting that TBB exhibits complicate behavior in regulating purinosomes—in the synchronized cells it largely acts as a purinosome disrupting agent, while in the normal cultured it also has certain purinosome promoting activity (see FIG. 2).

Also disclosed herein are methods of studying upstream signaling pathways that are involved in the purinosome formation. The disclosed methods can use the CK2 inhibitor induced DMR signal in native cells as a readout to identify its upstream pathways.

The present invention also discloses the use of purinosome-disrupting CK2 inhibitors as anti-cancer therapy agents. Purinosome formation is important for de novo purine synthesis, and purine synthesis pathway inhibitors are viable approach for anti-cancer therapy. Cancers that purine synthesis pathway inhibitors target include, but not limited to, leukemia, colorectal cancer, prostate cancer, breast cancer, and lymphoma, and particularly the cancer phenotype arising from the genetic alteration of onco-kinases (e.g. Abl and Alk). CK2 represents a “multi-purpose” target for the treatment of different kinds of tumors.

In one aspect, the present invention is directed to pharmaceutically acceptable salts of the purinsome disrupting CK2 inhibitors. As used herein, “pharmaceutically acceptable salts” includes salts of compounds of the present invention derived from the combination of such compounds with non-toxic acid or base addition salts.

Acid addition salts include inorganic acids such as hydrochloric, hydrobromic, hydroiodic, sulfuric, nitric and phosphoric acid, as well as organic acids such as acetic, citric, propionic, tartaric, glutamic, salicylic, oxalic, methanesulfonic, para-toluenesulfonic, succinic, and benzoic acid, and related inorganic and organic acids.

Base addition salts include those derived from inorganic bases such as ammonium and alkali and alkaline earth metal hydroxides, carbonates, bicarbonates, and the like, as well as salts derived from basic organic amines such as aliphatic and aromatic amines, aliphatic diamines, hydroxy alkamines, and the like. Such bases useful in preparing the salts of this invention thus include ammonium hydroxide, potassium carbonate, sodium bicarbonate, calcium hydroxide, methylamine, diethylamine, ethylenediamine, cyclohexylamine, ethanolamine and the like.

In addition to pharmaceutically-acceptable salts, other salts are included in the invention. They can serve as intermediates in the purification of the compounds, in the preparation of other salts, or in the identification and characterization of the compounds or intermediates.

The pharmaceutically acceptable salts of compounds of the present invention can also exist as various solvates, such as with water, methanol, ethanol, dimethylformamide, ethyl acetate and the like. Mixtures of such solvates can also be prepared. The source of such solvate can be from the solvent of crystallization, inherent in the solvent of preparation or crystallization, or adventitious to such solvent. Such solvates are within the scope of the present invention.

The present invention also encompasses the pharmaceutically acceptable prodrugs of the purinsome disrupting CK2 inhibitors. As used herein, “prodrug” is intended to include any compounds which are converted by metabolic processes within the body of a subject to an active agent that has a formula within the scope of the present invention. Since prodrugs are known to enhance numerous desirable qualities of pharmaceuticals (e.g., solubility, bioavailability, manufacturing, etc.) the compounds of the present invention can be delivered in prodrug form. Conventional procedures for the selection and preparation of suitable prodrug derivatives are described, for example, in Prodrugs, Sloane, K. B., Ed.; Marcel Dekker: New York, 1992, incorporated by reference herein in its entirety.

It is recognized that compounds of the present invention can exist in various stereoisomeric forms. As such, the compounds of the present invention notably include geometric isomers of (Z) or (E) configurations and optical isomers.

The purinsome-disrupting CK2 inhibitors are advantageously used as active ingredient, in particular, of anticancer, antiviral or anti-inflammatory medicaments.

Preferably, said compounds can be used to treat and/or prevent the cancers selected from breast, prostate, squamous cell carcinoma of head and neck, lung, brain or glioma cancer.

Alternatively, said compounds can be used to prevent and/or to treat viral diseases and disorders notably selected from Herpes, Cytomegalovirus, or inflammatory diseases and disorders, notably chronic inflammatory disorders (in particular chronic intestinal inflammation), or glomerulonephritis.

In particular, the purinsome-disrupting CK2 inhibitors can be used for the treatment and/or prevention of pathology associated with a deregulated (i.e. increased or decreased) activity of CK2 in cells.

According to a still further object, the present invention is also concerned with the use of a purinsome-disrupting CK2 inhibitor for the preparation of a medicament intended for the prevention and/or treatment of cancer, viral or inflammatory diseases and disorders.

The present invention also concerns the corresponding methods of treatment comprising the administration of a therapeutically effective amount of a compound of the invention together with a pharmaceutically acceptable carrier or recipient to a patient in the need thereof.

The identification of those subjects who are in need of treatment of herein-described diseases and conditions is well within the ability and knowledge of one skilled in the art. A clinician skilled in the art can readily identify, by the use of clinical tests, physical examination and medical/family history, those subjects who are in need of such treatment.

Disclosed are methods for testing a molecule comprising, a) incubating the molecule with a cell system comprising a multienzyme complex forming an incubated cell system, b) assaying the incubated cell system with a label free biosensor system. c) measuring the ability of the molecule to modulate a the multi-enzyme complex, and/or alone or in any combination with any step, reagent, compound, molecule, agent, or composition disclosed herein.

Also disclosed are methods, further comprising the step of classifying the molecule as a multienzyme complex modulator, wherein the cell system comprises synchronized cells, where the synchronization of the cells comprises culturing the cells in a purine rich serum medium with a high initial seeding number such that the cells once attached reach high confluency; culturing cells in serum but purine depleted medium; or culturing the cells of a highly confluency under long term starvation conditions, wherein the cells are synchronized to have a basal level of multienzyme complex activity, wherein the multienzyme complex modulator comprises a multienzyme disassembly promoting agent, wherein the multienzyme disassembly promoting agent comprises a purinosome complex disrupting agent, wherein the purinosome complex disrupting agent comprises a purinosome disrupting CK2 inhibitor, wherein the purinosome disrupting CK2 inhibitor comprises TBB, wherein the purinosome disrupting CK2 inhibitor is a purine synthesis pathway inhibitor, wherein the multienzyme complex modulator comprises a multienzyme complex promoting agent, wherein the multienzyme complex promoting agent comprises a purinosome complex promoting agent, wherein the purinosome complex promoting agent comprises a purinosome promoting CK2 inhibitor, wherein the purinosome promoting CK2 inhibitor comprises DMAT, apigenin, DRB, or TBCA, wherein the multienzyme complex is involved in purine synthesis, wherein the cell system comprises live cells having CK2 activity, and/or alone or in any combination with any step, reagent, compound, molecule, agent, or composition disclosed herein.

Also disclosed are methods further comprising the step of incubating the cells with a reference probe, wherein the reference probe comprises a purinsome complex promoting agent, wherein the purinosome complex promoting agent comprises DMAT, apigenin, DRB, or TBCA, wherein the reference probe comprises a purinosome complex disrupting agent, wherein the purinosome complex promoting agent comprises TBB, and/or alone or in any combination with any step, reagent, compound, molecule, agent, or composition disclosed herein.

Also disclosed are methods, wherein the step of classifying comprises comparing a molecule induced biosensor response with a reference probe biosensor response wherein the similarity in the biosensor responses is an indicator that the molecule is classified in the same class as the reference probe, wherein the reference probe comprises a CK2 inhibitor, wherein the biosensor response comprises a DMR signal, wherein the step of comparing comprises assaying the molecule against both a purinosome complex disrupting agent and a purinosome promoting agent, wherein the purinosome complex disrupting agent comprises TBB and the purinosome promoting agent comprises DMAT, wherein a molecule that results in a DMR signal similar to TBB, and selectively inhibits the TBB DMR signal but potentiates the DMAT DMR signal is a purinosome disrupting agent, wherein a molecule that results in a DMR signal similar to DMAT, and selectively inhibits the DMAT DMR signal but potentiates the TBB DMR signal is a purinosome promoting agent, wherein the step of comparing comprises comparing a molecule-induced DMR index with a known CK2 inhibitor DMR index acting on a panel of CK2 inhibitor sensitive cells, wherein the similarity between the two indices is an indicator that the molecule is a CK2 inhibitor, wherein the step of classifying comprises generating DMR modulation indices of both a molecule and a known CK2 inhibitor against a panel of cells/markers, wherein the similarity between the two indices is an indicator that the molecule is a CK2 inhibitor, further comprising assaying the sensitivity of the molecule induced DMR signal to the preactivation of an endogenous Gi-coupled receptor, wherein the molecule induced DMR that is similar to TBB, and is potentiated in the cell system having the preactivation of the Gi-coupled receptor is an indicator that the molecule is purinosome disrupting CK2 inhibitor, and/or alone or in any combination with any step, reagent, compound, molecule, agent, or composition disclosed herein.

Also disclosed are methods of treating cancer in a subject comprising administering a purinosome disrupting CK2 inhibitor, and/or alone or in any combination with any step, reagent, compound, molecule, agent, or composition disclosed herein.

Also disclosed are methods, wherein the cancer is leukemia, colorectal cancer, prostate cancer, breast cancer, and lymphoma, squamous cell carcinoma of head and neck, lung, brain glioma cancer or a cancer phenotype related to abnormal activity of CK2 kinase, wherein the purinosome disrupting CK2 inhibitor comprises a pharmaceutically acceptable salt of the purinsome disrupting CK2 inhibitor, and/or alone or in any combination with any step, reagent, compound, molecule, agent, or composition disclosed herein.

Disclosed are methods of treating a viral infection in a subject comprising administering a purinosome disrupting CK2 inhibitor, and/or alone or in any combination with any step, reagent, compound, molecule, agent, or composition disclosed herein.

Also disclosed are methods, wherein the viral infection comprises a Herpes virus or Cytomegalovirus, and/or alone or in any combination with any step, reagent, compound, molecule, agent, or composition disclosed herein, and/or alone or in any combination with any step, reagent, compound, molecule, agent, or composition disclosed herein.

Disclosed are methods of treating an inflammation condition in a subject comprising administering a purinosome disrupting CK2 inhibitor.

Also disclosed are methods, wherein the inflammatory condition comprises a chronic inflammation disorder related to abnormal activity of CK2 kinase, wherein the chronic inflammation disorder is an intestinal disorder or glomerulonephritis, wherein the intestinal disorder is inflammatory bowel disease, ulcerative colitis, or Crohn's disease, wherein the composition is administered in a therapeutically effective amount of the purinosome disrupting CK2 inhibitor, wherein the subject is in need of treatment with the purinosome disrupting CK2 inhibitor, and/or alone or in any combination with any step, reagent, compound, molecule, agent, or composition disclosed herein.

1. Biosensors and Biosensor Assays

Label-free cell-based assays generally employ a biosensor to monitor molecule-induced responses in living cells. The molecule can be naturally occurring or synthetic, and can be a purified or unpurified mixture. A biosensor typically utilizes a transducer such as an optical, electrical, calorimetric, acoustic, magnetic, or like transducer, to convert a molecular recognition event or a molecule-induced change in cells contacted with the biosensor into a quantifiable signal. These label-free biosensors can be used for molecular interaction analysis, which involves characterizing how molecular complexes form and disassociate over time, or for cellular response, which involves characterizing how cells respond to stimulation. The biosensors that are applicable to the present methods can include, for example, optical biosensor systems such as surface plasmon resonance (SPR) and resonant waveguide grating (RWG) biosensors, resonant mirrors, ellipsometers, and electric biosensor systems such as bioimpedance systems, and the variants of these biosensors.

i. SPR Biosensors and Systems

SPR relies on a prism to direct a wedge of polarized light, covering a range of incident angles, into a planar glass substrate bearing an electrically conducting metallic film (e.g., gold) to excite surface plasmons. The resultant evanescent wave interacts with, and is absorbed by, free electron clouds in the gold layer, generating electron charge density waves (i.e., surface plasmons) and causing a reduction in the intensity of the reflected light. The resonance angle at which this intensity minimum occurs is a function of the refractive index of the solution close to the gold layer on the opposing face of the sensor surface

ii. RWG Biosensors and Systems

An RWG biosensor can include, for example, a substrate (e.g., glass), a waveguide thin film with an embedded grating or periodic structure, and a cell layer. The RWG biosensor utilizes the resonant coupling of light into a waveguide by means of a diffraction grating, leading to total internal reflection at the solution-surface interface, which in turn creates an electromagnetic field at the interface. This electromagnetic field is evanescent in nature, meaning that it decays exponentially from the sensor surface; the distance at which it decays to 1/e of its initial value is known as the penetration depth and is a function of the design of a particular RWG biosensor, but is typically on the order of about 200 nm. This type of biosensor exploits such evanescent wave to characterize ligand-induced alterations of a cell layer at or near the sensor surface.

RWG instruments can be subdivided into systems based on angle-shift or wavelength-shift measurements. In a wavelength-shift measurement, polarized light covering a range of incident wavelengths with a constant angle is used to illuminate the waveguide; light at specific wavelengths is coupled into and propagates along the waveguide. Alternatively, in angle-shift instruments, the sensor is illuminated with monochromatic light and the angle at which the light is resonantly coupled is measured.

The resonance conditions are influenced by the cell layer (e.g., cell confluency, adhesion and status), which is in direct contact with the surface of the biosensor. When a ligand or an analyte interacts with a cellular target (e.g., CK2) in living cells, any change in local refractive index within the cell layer can be detected as a shift in resonant angle (or wavelength).

The Corning® Epic® system uses RWG Biosensors for label-free biochemical or cell-based assays (Corning Inc., Corning, N.Y.). The Epic® System consists of an RWG plate reader and SBS (Society for Biomolecular Screening) standard microtiter plates. The detector system in the plate reader exploits integrated fiber optics to measure the shift in wavelength of the incident light, as a result of ligand-induced changes in the cells. A series of illumination-detection heads are arranged in a linear fashion, so that reflection spectra are collected simultaneously from each well within a column of a 384-well microplate. The whole plate is scanned so that each sensor can be addressed multiple times, and each column is addressed in sequence. The wavelengths of the incident light are collected and used for analysis. A temperature-controlling unit can be included in the instrument to minimize spurious shifts in the incident wavelength due to the temperature fluctuations. The measured response represents an averaged response of a population of cells. Varying features of the systems can be automated, such as sample loading, and can be multiplexed, such as with a 96 or 386 well microtiter plate. Liquid handling is carried out by either on-board liquid handler, or an external liquid handling accessory. Specifically, molecule solutions are directly added or pipetted into the wells of a cell assay plate having cells cultured in the bottom of each well. The cell assay plate contains certain volume of assay buffer solution covering the cells. A simple mixing step by pipetting up and down certain times can also be incorporated into the molecule addition step.

iii. Electrical Biosensors and Systems

Electrical biosensors consist of a substrate (e.g., plastic), an electrode, and a cell layer. In this electrical detection method, cells are cultured on small gold electrodes arrayed onto a substrate, and the system's electrical impedance is followed with time. The impedance is a measure of changes in the electrical conductivity of the cell layer. Typically, a small constant voltage at a fixed frequency or varied frequencies is applied to the electrode or electrode array, and the electrical current through the circuit is monitored over time. The ligand-induced change in electrical current provides a measure of cell response. Impedance measurement for whole cell sensing was first realized in 1984. Since then, impedance-based measurements have been applied to study a wide range of cellular events, including cell adhesion and spreading, cell micromotion, cell morphological changes, and cell death. Classical impedance systems suffer from high assay variability due to use of a small detection electrode and a large reference electrode. To overcome this variability, the latest generation of systems, such as the CellKey system (MDS Sciex, South San Francisco, Calif.) and RT-CES (ACEA Biosciences Inc., San Diego, Calif.), utilize an integrated circuit having a microelectrode array.

iv. High Spatial Resolution Biosensor Imaging Systems

Optical biosensor imaging systems, including SPR imaging systems, ellipsometry imaging systems, and RWG imaging systems, offer high spatial resolution, and can be used in embodiments of the disclosure. For example, SPR imager®II (GWC Technologies Inc) uses prism-coupled SPR, and takes SPR measurements at a fixed angle of incidence, and collects the reflected light with a CCD camera. Changes on the surface are recorded as reflectivity changes. Thus, SPR imaging collects measurements for all elements of an array simultaneously.

A swept wavelength optical interrogation system based on RWG biosensor for imaging-based application can be employed. In this system, a fast tunable laser source is used to illuminate a sensor or an array of RWG biosensors in a microplate format. The sensor spectrum can be constructed by detecting the optical power reflected from the sensor as a function of time as the laser wavelength scans, and analysis of the measured data with computerized resonant wavelength interrogation modeling results in the construction of spatially resolved images of biosensors having immobilized receptors or a cell layer. The use of an image sensor naturally leads to an imaging based interrogation scheme. 2 dimensional label-free images can be obtained without moving parts.

Alternatively, angular interrogation system with transverse magnetic or p-polarized TM₀ mode can also be used. This system consists of a launch system for generating an array of light beams such that each illuminates a RWG sensor with a dimension of approximately 200 μm×3000 μm or 200 μm×2000 μm, and a CCD camera-based receive system for recording changes in the angles of the light beams reflected from these sensors. The arrayed light beams are obtained by means of a beam splitter in combination with diffractive optical lenses. This system allows up to 49 sensors (in a 7×7 well sensor array) to be simultaneously sampled at every 3 seconds, or up to the whole 384 well microplate to be simultaneously sampled at every 10 seconds.

Alternatively, a scanning wavelength interrogation system can also be used. In this system, a polarized light covering a range of incident wavelengths with a constant angle is used to illuminate and scan across a waveguide grating biosensor, and the reflected light at each location can be recorded simultaneously. Through scanning, a high resolution image across a biosensor can also be achieved

v. Dynamic Mass Redistribution (DMR) Signals in Living Cells

The cellular response to stimulation through a cellular target can be encoded by the spatial and temporal dynamics of downstream signaling networks. For this reason, monitoring the integration of cell signaling in real time can provide physiologically relevant information that is useful in understanding cell biology and physiology.

Optical biosensors including resonant waveguide grating (RWG) biosensors, can detect an integrated cellular response related to dynamic redistribution of cellular matters, thus providing a non-invasive means for studying cell signaling. All optical biosensors are common in that they can measure changes in local refractive index at or very near the sensor surface. In principle, almost all optical biosensors are applicable for cell sensing, as they can employ an evanescent wave to characterize ligand-induced change in cells. The evanescent-wave is an electromagnetic field, created by the total internal reflection of light at a solution-surface interface, which typically extends a short distance (hundreds of nanometers) into the solution at a characteristic depth known as the penetration depth or sensing volume.

Recently, theoretical and mathematical models have been developed that describe the parameters and nature of optical signals measured in living cells in response to stimulation with ligands. These models, based on a 3-layer waveguide system in combination with known cellular biophysics, link the ligand-induced optical signals to specific cellular processes mediated through a receptor.

Because biosensors measure the average response of the cells located at the area illuminated by the incident light, a highly confluent layer of cells can be used to achieve optimal assay results. Due to the large dimension of the cells as compared to the short penetration depth of a biosensor, the sensor configuration is considered as a non-conventional three-layer system: a substrate, a waveguide film with a grating structure, and a cell layer. Thus, a ligand-induced change in effective refractive index (i.e., the detected signal) can be, to first order, directly proportional to the change in refractive index of the bottom portion of the cell layer:

ΔN=S(C)Δn_{c}

where S(C) is the sensitivity to the cell layer, and Δn_{c} the ligand-induced change in local refractive index of the cell layer sensed by the biosensor. Because the refractive index of a given volume within a cell is largely determined by the concentrations of bio-molecules such as proteins, Δn_{c} can be assumed to be directly proportional to ligand-induced change in local concentrations of cellular targets or molecular assemblies within the sensing volume. Considering the exponentially decaying nature of the evanescent wave extending away from the sensor surface, the ligand-induced optical signal is governed by:

$$Δ N = S ( C ) α d {\sum }_{i} Δ {C}_{i} [ {e}^{\frac{- {z}_{i}}{Δ {Z}_{C}}} - {e}^{\frac{- {z}_{i + 1}}{Δ {Z}_{C}}} ]$$

where ΔZ_{c} is the penetration depth into the cell layer, a the specific refraction increment (about 0.18/mL/g for proteins), zᵢ the distance where the mass redistribution occurs, and d an imaginary thickness of a slice within the cell layer. Here the cell layer is divided into an equal-spaced slice in the vertical direction. The equation above indicates that the ligand-induced optical signal is a sum of mass redistribution occurring at distinct distances away from the sensor surface, each with an unequal contribution to the overall response. Furthermore, the detected signal, in terms of wavelength or angular shifts, is primarily sensitive to mass redistribution occurring perpendicular to the sensor surface. Because of its dynamic nature, it also is referred to as dynamic mass redistribution (DMR) signal.

2. Cells and Biosensors

Cells rely on multiple cellular pathways or machineries to process, encode and integrate the information they receive. Unlike the affinity analysis with optical biosensors that specifically measures the binding of analytes to a protein target, living cells are much more complex and dynamic.

To study cell signaling, cells can be brought into contact with the surface of a biosensor, which can be achieved through cell culture. These cultured cells can be attached onto the biosensor surface through three types of contacts: focal contacts, close contacts and extracellular matrix contacts, each with its own characteristic separation distance from the surface. As a result, the basal cell membranes are generally located away from the surface by ˜10-100 nm. For suspension cells, the cells can be brought to contact with the biosensor surface through either covalent coupling of cell surface receptors, or specific binding of cell surface receptors, or simply settlement by gravity force. For this reason, biosensors are able to sense the bottom portion of cells.

Cells, in many cases, exhibit surface-dependent adhesion and proliferation. In order to achieve robust cell assays, the biosensor surface can require a coating to enhance cell adhesion and proliferation. However, the surface properties can have a direct impact on cell biology. For example, surface-bound ligands can influence the response of cells, as can the mechanical compliance of a substrate material, which dictates how it will deform under forces applied by the cell. Due to differing culture conditions (time, serum concentration, confluency, etc.), the cellular status obtained can be distinct from one surface to another, and from one condition to another. Thus, special efforts to control cellular status can be necessary in order to develop biosensor-based cell assays.

Cells are dynamic objects with relatively large dimensions—typically in the range of tens of microns. Even without stimulation, cells constantly undergo micromotion—a dynamic movement and remodeling of cellular structure, as observed in tissue culture by time lapse microscopy at the sub-cellular resolution, as well as by bio-impedance measurements at the nanometer level.

Under un-stimulated conditions cells generally produce an almost net-zero DMR response as examined with a RWG biosensor. This is partly because of the low spatial resolution of optical biosensors, as determined by the large size of the laser spot and the long propagation length of the coupled light. The size of the laser spot determines the size of the area studied—and usually only one analysis point can be tracked at a time. Thus, the biosensor typically measures an averaged response of a large population of cells located at the light incident area. Although cells undergo micromotion at the single cell level, the large populations of cells give rise to an average net-zero DMR response. Furthermore, intracellular macromolecules are highly organized and spatially restricted to appropriate sites in mammalian cells. The tightly controlled localization of proteins on and within cells determines specific cell functions and responses because the localization allows cells to regulate the specificity and efficiency of proteins interacting with their proper partners and to spatially separate protein activation and deactivation mechanisms. Because of this control, under un-stimulated conditions, the local mass density of cells within the sensing volume can reach an equilibrium state, thus leading to a net-zero optical response. In order to achieve a consistent optical response, the cells examined can be cultured under conventional culture conditions for a period of time such that most of the cells have just completed a single cycle of division.

Living cells have exquisite abilities to sense and respond to exogenous signals. Cell signaling was previously thought to function via linear routes where an environmental cue would trigger a linear chain of reactions resulting in a single well-defined response. However, research has shown that cellular responses to external stimuli are much more complicated. It has become apparent that the information that cells receive can be processed and encoded into complex temporal and spatial patterns of phosphorylation and topological relocation of signaling proteins. The spatial and temporal targeting of proteins to appropriate sites can be crucial to regulating the specificity and efficiency of protein-protein interactions, thus dictating the timing and intensity of cell signaling and responses. Pivotal cellular decisions, such as cytoskeletal reorganization, cell cycle checkpoints and apoptosis, depend on the precise temporal control and relative spatial distribution of activated signal-transducers. Thus, cell signaling mediated through a cellular target such as G protein-coupled receptor (GPCR) typically proceeds in an orderly and regulated manner, and consists of a series of spatial and temporal events, many of which lead to changes in local mass density or redistribution in local cellular matters of cells. These changes or redistribution, when occurring within the sensing volume, can be followed directly in real time using optical biosensors

i. DMR Signals as a Physiological Response of Living Cells

Through comparison with conventional pharmacological approaches for studying receptor biology, it has been shown that when a ligand is specific to a receptor expressed in a cell system, the ligand-induced DMR signal is receptor-specific, dose-dependent and saturate-able. In addition, the biosensor can distinguish full agonists, partial agonists, inverse agonists, antagonists, and allosteric modulators. Thus, DMR is capable of monitoring physiological responses of living cells.

ii. DMR Signals Contain Systems Cell Pharmacology Information of a Ligand Acting on Living Cells.

Since the DMR signal is an integrated cellular response consisting of contributions of many cellular events involving dynamic redistribution of cellular matters within the bottom portion of cells, a ligand-induced biosensor signal, such as a DMR signal contains systems cell pharmacology information. It is known that GPCRs often display rich behaviors in cells, and that many ligands can induce operative bias to favor specific portions of the cell machinery and exhibit pathway-biased efficacies. Thus, it is highly possibly that a ligand can have multiple efficacies, depending on how cellular events downstream of the receptor are measured and used as readout(s) for the ligand pharmacology. It is difficult in practice for conventional cell assays, which are mostly pathway-biased and assay only a single signaling event, to systematically represent the signaling potentials of GPCR ligands. However, because label-free biosensors cellular assays do not require prior knowledge of cell signaling, and are pathway-unbiased and pathway-sensitive, these biosensor cellular assays are amenable to studying ligand-selective signaling as well as systems cell pharmacology of any ligands.

iii. Biosensor Parameters

A label-free biosensor such as RWG biosensor or bioimpedance biosensor is able to follow in real time ligand-induced cellular response. The non-invasive and manipulation-free biosensor cellular assays do not require prior knowledge of cell signaling. The resultant biosensor signal contains high information relating to receptor signaling and ligand pharmacology. Multi-parameters can be extracted from the kinetic biosensor response of cells upon stimulation. These parameters include, but not limited to, the overall dynamics (e.g., shape, oscillation patterns, and durations), phases, signal amplitudes, as well as kinetic parameters including the transition time from one phase to another, and the kinetics of each phase (see Fang, Y., and Ferrie, A. M. (2008) “label-free optical biosensor for ligand-directed functional selectivity acting on β2 adrenoceptor in living cells”. FEBS Lett. 582, 558-564; Fang, Y., et al., (2005) “Characteristics of dynamic mass redistribution of EGF receptor signaling in living cells measured with label free optical biosensors”. Anal. Chem., 77, 5720-5725; Fang, Y., et al., (2006) “Resonant waveguide grating biosensor for living cell sensing”. Biophys. J., 91, 1925-1940).

3. Cell Synchronization

The cell synchronization that renders CK2 activity for being measured robustly can be achieved through at least three means, which can be used independently or in any combination of ways.

i. Culturing with Initial High Seeding Numbers of Cells

In one method, the cells are synchronized through culturing. Here high initial seeding numbers of cells are used such that the cells reach high confluency early (i.e., right after attachment). In this method, cells are cultured under serum containing and purine rich medium for over night, (such as at least 12 hours). For example, HeLa cells can form a monolayer (i.e., confluency of >90%) when an initial seeding number is as less as 10000 cells per well for a 384 well microplate, and the cells undergo overnight culture. However, cells do not reach high confluency right after the attachment. Cell attachment is a rapid process and typically much shorter (e.g., 1 hr to 6 hrs) than the cell duplication time. On the other hand, HeLa cells will reach high confluency right after attachment, when the initial seeding numbers are greater than 20 k per well in a 384 well microplate.

ii. Ultra-High Confluency Culturing

In one method, the cells are synchronized through culturing. Here high initial seeding numbers of cells with serum-rich and purine-rich medium are used such that the cells reach high confluency early (i.e., the time being close to a single cycle of cell duplication which is typically within 16 hrs to 60 hrs), and undergo quiescence through continuous culture in either serum rich medium or in serum-free medium for an extended period of time (typically overnight). During such culture condition, cells reach ultra high confluency (>99%), and are in a fully quiescent state via the combination of contact inhibition and serum withdrawal.

iii. Culturing with Purine Depleted Medium

In another method, the cells are synchronized through incubation in a specific buffered solution. Here regular seeding numbers of cells can be used to culture cells onto the sensor surface using a serum-rich but purine depleted medium until it reach high confluency (>90%).

### C. DEFINITIONS

Various embodiments of the disclosure will be described in detail with reference to drawings, if any. Reference to various embodiments does not limit the scope of the disclosure, which is limited only by the scope of the claims attached hereto. Additionally, any examples set forth in this specification are not intended to be limiting and merely set forth some of the many possible embodiments for the claimed invention.

1. A

As used in the specification and the appended claims, the singular forms “a,” “an” and “the” or like terms include plural referents unless the context clearly dictates otherwise. Thus, for example, reference to “a pharmaceutical carrier” includes mixtures of two or more such carriers, and the like.

2. Abbreviations

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

3. About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

4. Agonism and Antagonism Mode

The agonism mode or like terms is the assay wherein the cells are exposed to a molecule to determine the ability of the molecule to trigger biosensor signals such as DMR signals, while the antagonism mode is the assay wherein the cells are exposed to a maker in the presence of a molecule to determine the ability of the molecule to modulate the biosensor signal of cells responding to the marker.

5. Analytical Methods

An analytical method is, for example, a method which measures a molecule or substance. For example, gas chromatography, gel permeation chromatography, high resolution gas chromoatography, high resolution mass spectrometry, or mass spectrometry is analytical methods.

6. Assaying

Assaying, assay, or like terms refers to an analysis to determine a characteristic of a substance, such as a molecule or a cell, such as for example, the presence, absence, quantity, extent, kinetics, dynamics, or type of an a cell's optical or bioimpedance response upon stimulation with one or more exogenous stimuli, such as a ligand or marker. Producing a biosensor signal of a cell's response to a stimulus can be an assay.

7. Assaying the Response

“Assaying the response” or like terms means using a means to characterize the response. For example, if a molecule is brought into contact with a cell, a biosensor can be used to assay the response of the cell upon exposure to the molecule.

8. Association and Dissociation of Purinosomes in Real Time

Assaying the association and dissociation of purinosomes in real time refers to monitoring the association and dissociation of the purinosomes as the association and dissociation are occurring.

9. Biosensor

Biosensor or like terms refer to a device for the detection of an analyte that combines a biological component with a physicochemical detector component. The biosensor typically consists of three parts: a biological component or element (such as tissue, microorganism, pathogen, cells, or combinations thereof), a detector element (works in a physicochemical way such as optical, piezoelectric, electrochemical, thermometric, or magnetic), and a transducer associated with both components. The biological component or element can be, for example, a living cell, a pathogen, or combinations thereof. In embodiments, an optical biosensor can comprise an optical transducer for converting a molecular recognition or molecular stimulation event in a living cell, a pathogen, or combinations thereof into a quantifiable signal.

10. Biosensor Response

A “biosensor response”, “biosensor output signal”, “biosensor signal” or like terms is any reaction of a sensor system having a cell to a cellular response. A biosensor converts a cellular response to a quantifiable sensor response. A biosensor response is an optical response upon stimulation as measured by an optical biosensor such as RWG or SPR or it is a bioimpedence response of the cells upon stimulation as measured by an electric biosensor. Since a biosensor response is directly associated with the cellular response upon stimulation, the biosensor response and the cellular response can be used interchangeably, in embodiments of disclosure.

11. Biosensor Signal

A “biosensor signal” or like terms refers to the signal of cells measured with a biosensor that is produced by the response of a cell upon stimulation.

12. Cancer

leukemia, colorectal cancer, prostate cancer, breast cancer, and lymphoma, squamous cell carcinoma of head and neck, lung, brain glioma cancer or a cancer phenotype arising from the genetic alteration of onco-kinases such as Abl or Alk.

13. Cell

Cell or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

A cell can include different cell types, such as a cell associated with a specific disease, a type of cell from a specific origin, a type of cell associated with a specific target, or a type of cell associated with a specific physiological function. A cell can also be a native cell, an engineered cell, a transformed cell, an immortalized cell, a primary cell, an embryonic stem cell, an adult stem cell, a cancer stem cell, or a stem cell derived cell.

Human consists of about 210 known distinct cell types. The numbers of types of cells can almost unlimited, considering how the cells are prepared (e.g., engineered, transformed, immortalized, or freshly isolated from a human body) and where the cells are obtained (e.g., human bodies of different ages or different disease stages, etc).

14. Cell Culture

“Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also the culturing of complex tissues and organs.

15. Cell Panel

A “cell panel” or like terms is a panel which comprises at least two types of cells. The cells can be of any type or combination disclosed herein.

16. Cellular Response

A “cellular response” or like terms is any reaction by the cell to a stimulation.

17. Cellular Process

A cellular process or like terms is a process that takes place in or by a cell. Examples of cellular process include, but not limited to, proliferation, apoptosis, necrosis, differentiation, cell signal transduction, polarity change, migration, or transformation.

18. Cellular Target

A “cellular target” or like terms is a biopolymer such as a protein or nucleic acid whose activity can be modified by an external stimulus. Cellular targets are most commonly proteins such as enzymes, kinases, ion channels, and receptors.

19. Cell System

A cell system is any system of cells and reagents where the reagents are sufficient for culturing the cells, such as a growth medium.

20. Characterizing

Characterizing or like terms refers to gathering information about any property of a substance, such as a ligand, molecule, marker, or cell, such as obtaining a profile for the ligand, molecule, marker, or cell.

21. CK2 Modulator

A CK2 modulator is any agent that modulates a CK2 complex relative to a control.

22. CK2 Inhibitor

A CK2 inhibitor is any agent that inhibits, decreases, reduces, or prevents the enzymatic activity of CK2 kinase relative to a control.

23. CK2 Inhibitor Sensitive Cell

CK2 inhibitor sensitive cell is any cell or cell system that produces a detectable biosensor response when stimulated with a known CK2 inhibitor.

24. Chronic Inflammation Disorder or Condition

A chronic inflammation disorder or condition is any inflammation disorder or condition in which the inflammation disorder or condition in which new connective tissue is formed. For example, intestinal disorders, such as inflammatory bowel disease, ulcerative colitis, and Crohn's disease can be a chonic inflammation disorder or condition. Glomerulonephritis is also a chronic inflammation disorder.

25. Comprise

Throughout the description and claims of this specification, the word “comprise” and variations of the word, such as “comprising” and “comprises,” means “including but not limited to,” and is not intended to exclude, for example, other additives, components, integers or steps.

26. Consisting Essentially of

“Consisting essentially of in embodiments refers, for example, to a surface composition, a method of making or using a surface composition, formulation, or composition on the surface of the biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface modifier or condition, a particular ligand candidate, or like structure, material, or process variable selected. Items that may materially affect the basic properties of the components or steps of the disclosure or may impart undesirable characteristics to the present disclosure include, for example, decreased affinity of the cell for the biosensor surface, aberrant affinity of a stimulus for a cell surface receptor or for an intracellular receptor, anomalous or contrary cell activity in response to a ligand candidate or like stimulus, and like characteristics.

27. Components

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these molecules may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

28. Contacting

Contacting or like terms means bringing into proximity such that a molecular interaction can take place, if a molecular interaction is possible between at least two things, such as molecules, cells, markers, at least a compound or composition, or at least two compositions, or any of these with an article(s) or with a machine. For example, contacting refers to bringing at least two compositions, molecules, articles, or things into contact, i.e., such that they are in proximity to mix or touch For example, having a solution of composition A and cultured cell B and pouring solution of composition A over cultured cell B would be bringing solution of composition A in contact with cell culture B. Contacting a cell with a ligand would be bringing a ligand to the cell to ensure the cell have access to the ligand.

It is understood that anything disclosed herein can be brought into contact with anything else. For example, a cell can be brought into contact with a marker or a molecule, a biosensor, and so forth.

29. Compounds and Compositions

Compounds and compositions have their standard meaning in the art. It is understood that wherever, a particular designation, such as a molecule, substance, marker, cell, or reagent compositions comprising, consisting of, and consisting essentially of these designations are disclosed. Thus, where the particular designation marker is used, it is understood that also disclosed would be compositions comprising that marker, consisting of that marker, or consisting essentially of that marker. Where appropriate wherever a particular designation is made, it is understood that the compound of that designation is also disclosed. For example, if particular biological material, such as EGF, is disclosed EGF in its compound form is also disclosed.

30. Control

The terms control or “control levels” or “control cells” or like terms are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects or objects or reagents etc are treated as in a parallel experiment except for omission of the procedure or agent or variable etc under test and which is used as a standard of comparison in judging experimental effects. Thus, the control can be used to determine the effects related to the procedure or agent or variable etc. For example, if the effect of a test molecule on a cell was in question, one could a) simply record the characteristics of the cell in the presence of the molecule, b) perform a and then also record the effects of adding a control molecule with a known activity or lack of activity, or a control composition (e.g., the assay buffer solution (the vehicle)) and then compare effects of the test molecule to the control. In certain circumstances once a control is performed the control can be used as a standard, in which the control experiment does not have to be performed again and in other circumstances the control experiment should be run in parallel each time a comparison will be made.

31. Clathrate

A compound for use in the invention may form a complex such as a “clathrate”, a drug-host inclusion complex, wherein, in contrast to solvates, the drug and host are present in stoichiometric or non-stoichiometric amounts. A compound used herein can also contain two or more organic and/or inorganic components which can be in stoichiometric or non-stoichiometric amounts. The resulting complexes can be ionized, partially ionized, or non-ionized. For a review of such complexes, see J. Pharm. ScL, 64 (8), 1269-1288, by Haleblian (August 1975).

32. Detect

Detect or like terms refer to an ability of the apparatus and methods of the disclosure to discover or sense a molecule- or a marker-induced cellular response and to distinguish the sensed responses for distinct molecules.

33. Direct Action (of a Drug Candidate Molecule)

A “direct action” or like terms is a result (of a drug candidate molecule“) acting independently on a cell.

34. DMR Signal

A “DMR signal” or like terms refers to the signal of cells measured with an optical biosensor that is produced by the response of a cell upon stimulation.

35. DMR Response

A “DMR response” or like terms is a biosensor response using an optical biosensor. The DMR refers to dynamic mass redistribution or dynamic cellular matter redistribution. A P-DMR is a positive DMR response, a N-DMR is a negative DMR response, and a RP-DMR is a recovery P-DMR response.

36. DMAT

DMAT is 2-Dimethylamino-4,5,6,7-tetrabromo-1H-benzimidazole.

37. Disease Marker

A disease marker is any reagent, molecule, substance etc, that can be used for identifying, diagnosing, or prognosing is for the EGFR or VEGFR related disease.

38. Drug Candidate Molecule

A drug candidate molecule or like terms is a test molecule which is being tested for its ability to function as a drug or a pharmacophore. This molecule may be considered as a lead molecule.

39. Efficacy

Efficacy or like terms is the capacity to produce a desired size of an effect under ideal or optimal conditions. It is these conditions that distinguish efficacy from the related concept of effectiveness, which relates to change under real-life conditions. Efficacy is the relationship between receptor occupancy and the ability to initiate a response at the molecular, cellular, tissue or system level.

40. Higher and Inhibit and Like Words

The terms higher, increases, elevates, or elevation or like terms or variants of these terms, refer to increases above basal levels, e.g., as compared a control. The terms low, lower, reduces, decreases or reduction or like terms or variation of these terms, refer to decreases below basal levels, e.g., as compared to a control. For example, basal levels are normal in vivo levels prior to, or in the absence of, or addition of a molecule such as an agonist or antagonist to a cell Inhibit or forms of inhibit or like terms refers to to reducing or suppressing.

41. High Initial Seeding Number

High initial seeding number or the like term refer to a total numbers of cells seeded into a microtiter plate well that is relatively high, such that the cells reach high confluency (>90%) at time of either attachment (˜1 hr to 6 hr after seeding), or overnight culture (˜16-24 hrs).

42. High Confluency

Cell confluency or like terms refers to the coverage or proliferation that the cells are allowed over or throughout the culture medium. Since many types of cells can undergo cell contact inhibition, a high confluency means that the cells cultured reach high coverage (>90%) to form a cell monolayer on a tissue culture surface or a biosensor surface, and have significant restriction to the growth of the cells in the medium. Conversely, a low confluency (e.g., a confluency of 40-60%) means that there may be little or no restriction to the growth of the cells in/on the medium and they can be assumed to be in a growth phase.

43. Highly Confluent Cell

A highly confluent cell or the like term refers to a cell monolayer with a surface coverage of at least 90%.

44. In the Presence of the Molecule

“in the presence of the molecule” or like terms refers to the contact or exposure of the cultured cell with the molecule. The contact or exposure can be taken place before, or at the time, the stimulus is brought to contact with the cell.

45. Incubated Cell System

An incubated cell system is any cell system in which at least one agent, such as a molecule, has been added to the cell system.

46. Inflammation Condition

An inflammation condition is any condition that manifests with an increase in inflammation as a symptom or as a cause of the condition.

47. Index

An index or like terms is a collection of data. For example, an index can be a list, table, file, or catalog that contains one or more modulation profiles. It is understood that an index can be produced from any combination of data. For example, a DMR profile can have a P-DMR, a N-DMR, and a RP-DMR. An index can be produced using the completed date of the profile, the P-DMR data, the N-DMR data, the RP-DMR data, or any point within these, or in combination of these or other data. The index is the collection of any such information. Typically, when comparing indexes, the indexes are of like data, i.e. P-DMR to P-DMR data.

i. Biosensor Index

A “biosensor index” or like terms is an index made up of a collection of biosensor data. A biosensor index can be a collection of biosensor profiles, such as primary profiles, or secondary profiles. The index can be comprised of any type of data. For example, an index of profiles could be comprised of just an N-DMR data point, it could be a P-DMR data point, or both or it could be an impedence data point. It could be all of the data points associated with the profile curve.

ii. DMR Index

A “DMR index” or like terms is a biosensor index made up of a collection of DMR data.

48. Known Molecule

A known molecule or like terms is a molecule with known pharmacological/biological/physiological/pathophysiological activity whose precise mode of action(s) may be known or unknown.

49. Known Modulator

A known modulator or like terms is a modulator where at least one of the targets is known with a known affinity. For example, a known modulator could be a CK2 inhibitor, PI3K inhibitor, a PKA inhibitor, a GPCR antagonist, a GPCR agonist, a RTK inhibitor, an epidermal growth factor receptor neutralizing antibody, or a phosphodiesterase inhibition, a PKC inhibitor or activator, etc.

50. Known Modulator Biosensor Index

A “known modulator biosensor index” or like terms is a modulator biosensor index produced by data collected for a known modulator. For example, a known modulator biosensor index can be made up of a profile of the known modulator acting on the panel of cells, and the modulation profile of the known modulator against the panels of markers, each panel of markers for a cell in the panel of cells.

51. Known Modulator DMR Index

A “known modulator DMR index” or like terms is a modulator DMR index produced by data collected for a known modulator. For example, a known modulator DMR index can be made up of a profile of the known modulator acting on the panel of cells, and the modulation profile of the known modulator against the panels of markers, each panel of markers for a cell in the panel of cells.

52. Label Free Biosensor System

A label free biosensor system is any system, as described herein, which uses a biosensor in a label free manner, as described herein.

53. Ligand

A ligand or like terms is a substance or a composition or a molecule that is able to bind to and form a complex with a biomolecule to serve a biological purpose. Actual irreversible covalent binding between a ligand and its target molecule is rare in biological systems. Ligand binding to receptors alters the chemical conformation, i.e., the three dimensional shape of the receptor protein. The conformational state of a receptor protein determines the functional state of the receptor. The tendency or strength of binding is called affinity. Ligands include substrates, blockers, inhibitors, activators, and neurotransmitters. Radioligands are radioisotope labeled ligands, while fluorescent ligands are fluorescently tagged ligands; both can be considered as ligands are often used as tracers for receptor biology and biochemistry studies. Ligand and modulator are used interchangeably.

54. Library

A library or like terms is a collection. The library can be a collection of anything disclosed herein. For example, it can be a collection, of indexes, an index library; it can be a collection of profiles, a profile library; or it can be a collection of DMR indexes, a DMR index library; Also, it can be a collection of molecule, a molecule library; it can be a collection of cells, a cell library; it can be a collection of markers, a marker library; A library can be for example, random or non-random, determined or undetermined. For example, disclosed are libraries of DMR indexes or biosensor indexes of known modulators.

55. Long Term Starvation Conditions

A long term starvation condition or the like term refers to a culture condition that a monolayer of cells has undergone an extended and prolonged culture period that is at least 3× doubling time.

56. Marker

A marker or like terms is a ligand which produces a signal in a biosensor cellular assay. The signal is, must also be, characteristic of at least one specific cell signaling pathway(s) and/or at least one specific cellular process(es) mediated through at least one specific target(s). The signal can be positive, or negative, or any combinations (e.g., oscillation). An EGFR activator, such as EGF, can be a marker for A431 cells wherein EGFRs are stably expressed.

57. Marker Panel

A “marker panel” or like terms is a panel which comprises at least two markers. The markers can be for different pathways, the same pathway, different targets, or even the same targets.

58. Marker Biosensor Index

A “marker biosensor index” or like terms is a biosensor index produced by data collected for a marker. For example, a marker biosensor index can be made up of a profile of the marker acting on the panel of cells, and the modulation profile of the marker against the panels of markers, each panel of markers for a cell in the panel of cells.

59. Marker DMR Index

A “marker biosensor index” or like terms is a biosensor DMR index produced by data collected for a marker. For example, a marker DMR index can be made up of a profile of the marker acting on the panel of cells, and the modulation profile of the marker against the panels of markers, each panel of markers for a cell in the panel of cells.

60. Material

Material is the tangible part of something (chemical, biochemical, biological, or mixed) that goes into the makeup of a physical object.

61. Mimic

As used herein, “mimic” or like terms refers to performing one or more of the functions of a reference object. For example, a molecule mimic performs one or more of the functions of a molecule.

62. Modulate

To modulate, or forms thereof, means either increasing, decreasing, or maintaining a cellular activity mediated through a cellular target. It is understood that wherever one of these words is used it is also disclosed that it could be 1%, 5%, 10%, 20%, 50%, 100%, 500%, or 1000% increased from a control, or it could be 1%, 5%, 10%, 20%, 50%, or 100% decreased from a control.

63. Modulator

A modulator or like terms is a ligand that controls the activity of a cellular target. It is a signal modulating molecule binding to a cellular target, such as a target protein.

64. Modulation Comparison

A “modulation comparison” or like terms is a result of normalizing a primary profile and a secondary profile.

65. Modulator Biosensor Index

A “modulator biosensor index” or like terms is a biosensor index produced by data collected for a modulator. For example, a modulator biosensor index can be made up of a profile of the modulator acting on the panel of cells, and the modulation profile of the modulator against the panels of markers, each panel of markers for a cell in the panel of cells.

66. Modulator DMR Index

A “modulator DMR index” or like terms is a DMR index produced by data collected for a modulator. For example, a modulator DMR index can be made up of a profile of the modulator acting on the panel of cells, and the modulation profile of the modulator against the panels of markers, each panel of markers for a cell in the panel of cells.

67. Modulate the Biosensor Signal of a Marker

“Modulate the biosensor signal or like terms is to cause changes of the biosensor signal or profile of a cell in response to stimulation with a marker.

68. Modulate the DMR Signal

“Modulate the DMR signal or like terms is to cause changes of the DMR signal or profile of a cell in response to stimulation with a marker.

69. Molecule

As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts. A unique characteristic of a molecule is that it has to be dissolvable in a solvent, such as water, aqueous solution, an organic solvent such as dimethyl sulfoxide (DMSO).

70. Molecule Mixture

A molecule mixture or like terms is a mixture containing at least two molecules. The two molecules can be, but not limited to, structurally different (i.e., enantiomers), or compositionally different (e.g., protein isoforms, glycoform, or an antibody with different poly(ethylene glycol) (PEG) modifications), or structurally and compositionally different (e.g., unpurified natural extracts, or unpurified synthetic compounds).

71. Molecule Biosensor Index

A “molecule biosensor index” or like terms is a biosensor index produced by data collected for a molecule. For example, a molecule biosensor index can be made up of a profile of the molecule acting on the panel of cells, and the modulation profile of the molecule against the panels of markers, each panel of markers for a cell in the panel of cells.

72. Molecule DMR Index

A “molecule DMR index” or like terms is a DMR index produced by data collected for a molecule. For example, a molecule biosensor index can be made up of a profile of the molecule acting on the panel of cells, and the modulation profile of the molecule against the panels of markers, each panel of markers for a cell in the panel of cells.

73. Molecule Index

A “molecule index” or like terms is an index related to the molecule.

74. Molecule-Treated Cell

A molecule-treated cell or like terms is a cell that has been exposed to a molecule.

75. Molecule Modulation Index

A “molecule modulation index” or like terms is an index to display the ability of the molecule to modulate the biosensor output signals of the panels of markers acting on the panel of cells. The modulation index is generated by normalizing a specific biosensor output signal parameter of a response of a cell upon stimulation with a marker in the presence of a molecule against that in the absence of any molecule.

76. Molecule Pharmacology

Molecule pharmacology or the like terms refers to the systems cell biology or systems cell pharmacology or mode(s) of action of a molecule acting on a cell. The molecule pharmacology is often characterized by, but not limited, toxicity, ability to influence specific cellular process(es) (e.g., proliferation, differentiation, reactive oxygen species signaling), or ability to modulate a specific cellular target (e.g, PI3K, PKA, PKC, PKG, JAK2, MAPK, MEK2, or actin).

77. Molecule Induced Biosensor Response

A molecule induced biosensor response is any response or signal of a particular cell system as measured with a biosensor that is correlated with the presence of the molecule relative to a control.

78. Multienzyme Complex

A multienzyme complex is any complex of more than two enzymes where the complex has a function dependent on the individual activities of each member enzyme.

79. Multienzyme Complex Modulator

A multienzyme complex modulator is any molecule or agent that alters the status of a multienzyme complex relative to a control.

80. Multienzyme Complex Promoting Agent

A multienzyme complex promoting agent or assembly promoting agent or the like term is any molecule or agent that causes or increases the formation of a multienzyme complex relative to a control.

81. Multienzyme Complex Disassembly Promoting Agent

A multienzyme complex disassembly promoting agent or the like term is any molecule or agent that decreases, inhibits, reduces, or prevents the formation of a complex, such as a multienzyme complex relative to a control.

82. Multienzyme Complex Disrupting Agent

A multienzyme complex disrupting agent or the like term is any molecule or agent that causes or increases the disassembly (or dissociation) of an already formed multienzyme complex relative to a control.

83. Normalizing

Normalizing or like terms means, adjusting data, or a profile, or a response, for example, to remove at least one common variable. For example, if two responses are generated, one for a marker acting a cell and one for a marker and molecule acting on the cell, normalizing would refer to the action of comparing the marker-induced response in the absence of the molecule and the response in the presence of the molecule, and removing the response due to the marker only, such that the normalized response would represent the response due to the modulation of the molecule against the marker. A modulation comparison is produced by normalizing a primary profile of the marker and a secondary profile of the marker in the presence of a molecule (modulation profile).

84. Optional

“Optional” or “optionally” or like terms means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

85. Or

The word “or” or like terms as used herein means any one member of a particular list and also includes any combination of members of that list.

86. Optimizing

Optimizing refers to a process of making better or checking to see if it something or some process can be made better.

87. Profile

A profile or like terms refers to the data which is collected for a composition, such as a cell. A profile can be collected from a label free biosensor as described herein.

i. Primary Profile

A “primary profile” or like terms refers to a biosensor response or biosensor output signal or profile which is produced when a molecule contacts a cell. Typically, the primary profile is obtained after normalization of initial cellular response to the net-zero biosensor signal (i.e., baseline).

ii. Secondary Profile

A “secondary profile” or like terms is a biosensor response or biosensor output signal of cells in response to a marker in the presence of a molecule. A secondary profile can be used as an indicator of the ability of the molecule to modulate the marker-induced cellular response or biosensor response.

iii. Modulation Profile

A “modulation profile” or like terms is the comparison between a secondary profile of the marker in the presence of a molecule and the primary profile of the marker in the absence of any molecule. The comparison can be by, for example, subtracting the primary profile from secondary profile or subtracting the secondary profile from the primary profile or normalizing the secondary profile against the primary profile.

88. Panel

A panel or like terms is a predetermined set of specimens (e.g., markers, or cells, or pathways). A panel can be produced from picking specimens from a library.

89. Positive Control

A “positive control” or like terms is a control that shows that the conditions for data collection can lead to data collection.

90. Potentiate

Potentiate, potentiated or like terms refers to an increase of a specific parameter of a biosensor response of a marker in a cell caused by a molecule. By comparing the primary profile of a marker with the secondary profile of the same marker in the same cell in the presence of a molecule, one can calculate the modulation of the marker-induced biosensor response of the cells by the molecule. A positive modulation means the molecule to cause increase in the biosensor signal induced by the marker.

91. Potency

Potency or like terms is a measure of molecule activity expressed in terms of the amount required to produce an effect of given intensity. For example, a highly potent drug evokes a larger response at low concentrations. The potency is proportional to affinity and efficacy. Affinity is the ability of the drug molecule to bind to a receptor.

92. Prodrug

“Prodrug” or the like terms refers to compounds that when metabolized in vivo, undergo conversion to compounds having the desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester, ether or amide derivatives of the compounds herein, and their pharmaceutically acceptable salts. For further discussions of prodrugs, see e.g., T. Higuchi and V. Stella “Pro-drugs as Novel Delivery Systems,” ACS Symposium Series 14 (1975) and E. B. Roche ed., Bioreversible Carriers in Drug Design (1987).

93. Publications

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

94. Preactivation

Preactivation or the like term is a step or process wherein a cell or cell system is exposed to an agonist for a receptor such that the receptor becomes activated, and this activation occurs a specific period of times (minutes to hours, such as at least or less than or equal to 1, 2, 3, 4, 5, 6, 7, 8, 9, 10, 15, 20, 25, 30, 35, 40, 45, 50, 60, 120, 180, 240, 300, or 360 minutes) before the stimulation of the cells with another molecule.

95. Purine Depleted Medium

A purine depleted medium or the like term refers to a serum containing medium that have very little (e.g., less than 1 micromolar, or 100 nanomolar) or no purine molecules. The purine depleted medium can be made by dialysis of a serum containing medium over a period of time.

96. Purine Rich Serum Medium

Purine rich serum medium or the like term refer to a cell culture medium containing serum (e.g., a commonly commercially available serum such as fetal bovine serum, or heat-inactivated fetal bovine serum, or fetal calf serum, all of which generally contain purine molecules).

97. Purinosome Complex Disrupting Agent

A purinosome complex disrupting agent is any molecule or agent that decreases, inhibits, reduces, or prevents the formation of a purinosome complex, or increases or promotes the disassembly of an already formed purinosome complex relative to a control. An example is TBB.

98. Purinsome Inhibitor Agent

A purinosome inhibitor agent is any agent that inhibits, reduces, decreases, or prevents the function or activity or a puriosome relative to a control.

99. Purinosome Complex Promoting Agent

A purinosome complex promoting agent is any agent that increases or causes the formation of a purinosome complex or increases or stabilizes the formation of an already formed purinosome complex relative to a control. An example is DMAT.

100. Purinosome Promoting Agent

A purinosome promoting agent is any agent that increases or promotes the function or activity of a purinosome relative to a control.

101. Purinosome-Promoting CK2 Inhibitor

A purinosome promoting CK2 inhibitor is any CK2 inhibitor that causes or increases the formation of purinosome complexes relative to a control.

102. Purinsome Disrupting CK2 Inhibitor

A purinosome disrupting CK2 inhibitor is any CK2 inhibitor that causes or increases the dissociation of an already formed purinosome complexes, or inhibits, reduces, prevents or decreases the formation of purinosome complexes relative to a control.

103. Purine Synthesis Pathway Inhibitor

A purine synthesis pathway inhibitor is any agent that decreases, inhibits, reduces, or prevents a step in the purine synthesis pathway relative to a control.

104. Receptor

A receptor or like terms is a protein molecule embedded in either the plasma membrane or cytoplasm of a cell, to which a mobile signaling (or “signal”) molecule may attach. A molecule which binds to a receptor is called a “ligand,” and may be a peptide (such as a neurotransmitter), a hormone, a pharmaceutical drug, or a toxin, and when such binding occurs, the receptor goes into a conformational change which ordinarily initiates a cellular response. However, some ligands merely block receptors without inducing any response (e.g. antagonists). Ligand-induced changes in receptors result in physiological changes which constitute the biological activity of the ligands.

105. “Robust Biosensor Signal”

A “robust biosensor signal” is a biosensor signal whose amplitude(s) is significantly (such as 3×, 10×, 20×, 100×, or 1000×) above either the noise level, or the negative control response. The negative control response is often the biosensor response of cells after addition of the assay buffer solution (i.e., the vehicle). The noise level is the biosensor signal of cells without further addition of any solution. It is worthy of noting that the cells are always covered with a solution before addition of any solution.

106. “Robust DMR Signal”

A “robust DMR signal” or like terms is a DMR form of a “robust biosensor signal.”

107. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10″as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

108. Response

A response or like terms is any reaction to any stimulation.

109. Reference Probe

A reference probe or referencing probe or the like term is any agent that has a known or defined activity against a specific cellular target (e.g., CK2 kinase) in a particular cell system.

110. Reference Probe Biosensor Response

A reference probe biosensor response is any response or signal of a particular cell system as measured with a biosensor that is correlated with the presence of the reference probe relative to a control.

111. Sample

By sample or like terms is meant an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

112. Salt(s) and Pharmaceutically Acceptable Salt(s)

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formula I or II with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate. Furthermore, where the compounds of the invention carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (CrC₆) halides (e.g., methyl, ethyl, propyl, and butyl chlorides, bromides, and iodides), dialkyl sulfates (i.e., dimethyl, diethyl, dibuytl, and diamyl sulfates), long chain halides (i.e., decyl, lauryl, myristyl, and stearyl chlorides, bromides, and iodides), arylalkyl halides (i.e., benzyl and phenethyl bromides), and others.

In one embodiment, hemisalts of acids and bases may also be formed, for example, hemisulphate and hemicalcium salts.

The compounds of the invention and their salts may exist in both unsolvated and solvated forms.

113. “Selectively”

Selectively can be used with any attribute herein, such as inhibits or prevents or increases, and refers to a molecule that has at least '10× the activity in relation to a control.

114. Signaling Pathway(s)

A “defined pathway” or like terms is a path of a cell from receiving a signal (e.g., an exogenous ligand) to a cellular response (e.g., increased expression of a cellular target). In some cases, receptor activation caused by ligand binding to a receptor is directly coupled to the cell's response to the ligand. For example, the neurotransmitter GABA can activate a cell surface receptor that is part of an ion channel. GABA binding to a GABA A receptor on a neuron opens a chloride-selective ion channel that is part of the receptor. GABA A receptor activation allows negatively charged chloride ions to move into the neuron which inhibits the ability of the neuron to produce action potentials. However, for many cell surface receptors, ligand-receptor interactions are not directly linked to the cell's response. The activated receptor must first interact with other proteins inside the cell before the ultimate physiological effect of the ligand on the cell's behavior is produced. Often, the behavior of a chain of several interacting cell proteins is altered following receptor activation. The entire set of cell changes induced by receptor activation is called a signal transduction mechanism or pathway. The signaling pathway can be either relatively simple or quite complicated.

115. Similarity or Similarity of Indexes

“Similarity of indexes” or like terms is a term to express the similarity between two indexes, or among at least three indices, one for a molecule, based on the patterns of indices, and/or a matrix of scores. The matrix of scores are strongly related to their counterparts, such as the signatures of the primary profiles of different molecules in corresponding cells, and the nature and percentages of the modulation profiles of different molecules against each marker. For example, higher scores are given to more-similar characters, and lower or negative scores for dissimilar characters. Because there are only three types of modulation, positive, negative and neutral, found in the molecule modulation index, the similarity matrices are relatively simple. For example, a simple matrix will assign identical modulation (e.g., a positive modulation) a score of +1 and non-identical modulation a score of −1.

Alternatively, different scores can be given for a type of modulation but with different scales. For example, a positive modulation of 10%, 20%, 30%, 40%, 50%, 60%, 100%, 200%, etc, can be given a score of +1, +2, +3, +4, +5, +6, +10, +20, correspondingly. Conversely, for negative modulation, similar but in opposite score can be given. For example, a molecule I led to the following modulation pattern against a panel of makers in two different cells: EGF (epidermal growth factor) in A431 (P-DMR, −60%), EGF in A431 (N-DMR, −65%), EGF in HT29 (early P-DMR, −70%), EGF in HT29 (late P-DMR, −100%), MTX (mallotoxin) in HT29 (P-DMR, −60%), and NT (neurotensin) in HT29 (P-DMR, −106%). Thus, the score of the molecule I modulation index in coordination can be assigned as (−6, −6.5, −7, −10, −6, −10.6). Similarly, for another molecule II, its score in coordination is found to be (−9.2, −10, −10, −10, −6.2, −4.5). Thus, by comparing the scores between I and the second molecule II, one can conclude that both molecules possibly share a similar mode of action in the two cell lines examined, and act as an EGFR inhibitor (see U.S. application Ser. No. 12/623,693. Fang, Y. et al. “Methods for Characterizing Molecules”, Filed Nov. 23, 2009; U.S. application Ser. No. 12/623,708. Fang, Y. et al. “Methods of creating an index”, filed Nov. 23, 2009).

116. Solvate

The compounds herein, and the pharmaceutically acceptable salts thereof, may exist in a continuum of solid states ranging from fully amorphous to fully crystalline. They may also exist in unsolvated and solvated forms. The term “solvate” describes a molecular complex comprising the compound and one or more pharmaceutically acceptable solvent molecules (e.g., ethanol (EtOH)). The term “hydrate” is a solvate in which the solvent is water. Pharmaceutically acceptable solvates include those in which the solvent may be isotopically substituted (e.g., D₂O, d₆-acetone, d₆-DMSO).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will be the norm.

The compounds herein, and the pharmaceutically acceptable salts thereof, may also exist as multi-component complexes (other than salts and solvates) in which the compound and at least one other component are present in stoichiometric or non-stoichiomethc amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun., 17:1889-1896 (2004). For a general review of multi-component complexes, see J. K. Haleblian, J. Pharm. Sci. 64(8):1269-88 (1975).

117. Stable

When used with respect to pharmaceutical compositions, the term “stable” or like terms is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

118. Substance

A substance or like terms is any physical object. A material is a substance. Molecules, ligands, markers, cells, proteins, and DNA can be considered substances. A machine or an article would be considered to be made of substances, rather than considered a substance themselves.

119. Subject

As used throughout, by a subject or like terms is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) and mammals, non-human mammals, primates, non-human primates, rodents, birds, reptiles, amphibians, fish, and any other animal. In one aspect, the subject is a mammal such as a primate or a human. The subject can be a non-human.

120. Synchronized Cells

Synchronized cells or the like terms refer to a population of cells wherein the majority of cells in a single well of a microtiter plate are in the same state (e.g., the same cell cycle (such as G₀ or G₂), or a cell state having low basal purinosome complexes). Synchronize(d) cells or the like term can also refer to the manipulation of the environment surrounding the cells or the conditions at which cells are grown which results in a population of cells wherein most cells are in the same stage of the cell cycle, or in the same subcellular organization such as low basal purinosome complexes.

121. Test Molecule

A test molecule or like terms is a molecule which is used in a method to gain some information about the test molecule. A test molecule can be an unknown or a known molecule.

122. Treating

Treating or treatment or like terms can be used in at least two ways. First, treating or treatment or like terms can refer to administration or action taken towards a subject. Second, treating or treatment or like terms can refer to mixing any two things together, such as any two or more substances together, such as a molecule and a cell. This mixing will bring the at least two substances together such that a contact between them can take place.

When treating or treatment or like terms is used in the context of a subject with a disease, it does not imply a cure or even a reduction of a symptom for example. When the term therapeutic or like terms is used in conjunction with treating or treatment or like terms, it means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

123. Trigger

A trigger or like terms refers to the act of setting off or initiating an event, such as a response.

124. TBB

TBB is 4,5,6,7-tetrabromobenzotriazole.

125. TBBz

TBBz is 4,5,6,7-tetrabromobenzimidazole. 126. Therapeutically Effective Amount

The term therapeutically effective means that the amount of the composition used is of sufficient quantity to ameliorate one or more causes or symptoms of a disease or disorder. Such amelioration only requires a reduction or alteration or decrease, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

127. Therapeutic Efficacy

Therapeutic efficacy refers to the degree or extent of results from a treatment of a subject.

128. Toxicity Marker

A toxicity marker is any reagent, molecule, substance etc. that can be used for identifying, diagnosing, prognosing a level of toxicity of a substance, in, for example, an organism or cell or tissue or organ.

129. Values

Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

Thus, the disclosed methods, compositions, articles, and machines, can be combined in a manner to comprise, consist of, or consist essentially of, the various components, steps, molecules, and composition, and the like, discussed herein. They can be used, for example, in methods for characterizing a molecule including a ligand as defined herein; a method of producing an index as defined herein; or a method of drug discovery as defined herein.

130. Viral Infection

A viral infection is any infection arising from a virus. For example, a viral infection can be caused by a Herpes virus or Cytomegalovirus.

131. Unknown Molecule

An unknown molecule or like terms is a molecule with unknown biological/pharmacological/physiological/pathophysiological activity, but with known or unknown chemical structure.

### D. REFERENCES

WO2006108183 A2. Fang, Y., Ferrie, A. M., Fontaine, N. M., Yuen, P. K. and Lahiri, J. “Optical biosensors and cells”

An, S. et al. Reversible compartmentalization of de novo purine biosynthetic complexes in living cells. Science 2008, 320: 103-106

### E. EXAMPLES

### 1. Experimental Procedures

i. Materials

Epinephrine, clonidine, pinacidil, dopamine, acetylcholine, cytochalasin B, U-73122, phalloidin, vinblastine, nocodazole, TBB (4,5,6,7-tetrabromobenzotriazole), DMAT (2-Dimethylamino-4,5,6,7-tetrabromo-1H-benzimidazole), TBBz (4,5,6,7-tetrabromobenzimidazole), DRB (5,6-Dichlorobenzimidazole 1-(β-D-ribofuranoside), TBCA (Tetrabromocinnamic Acid), azaserine or hypoxanthine were obtained from Sigma Chemical Co. (St. Louis, Mo.). Epic® 384 biosensor microplates were obtained from Corning Inc. (Corning, N.Y.). Sphingosine-1-phosphate (S1P), lysophosphatidic Acid (LPA), ACEA, terbutaline, and oxymetazoline were obtained from Tocris (St. Louis, Mo.).

ii. Cell Culture

HeLa cells were obtained from American Type Cell Culture (Manassas, Va.). This cervical cancer cell line was maintained in standard serum medium (i.e., minimum essential medium (MEM) (Invitrogen) supplemented with 10% fetal bovine serum (FBS) and 1% Penicillin/streptomycin). To investigate the effects of purine depletion on the purinosome formation and dissociation, HeLa cells were maintained for at least two passages in “purine-depleted medium” prior cell assays. The purine depleted medium is a medium consisting of Roswell Park Memorial Institute 1640 (RPMI 1640) supplemented with dialyzed 5% FBS and. FBS was dialyzed against 0.9% NaCl at 4° C. for ˜2 days using a 25 kDa MWCO dialysis membrane to remove purines. This process completely removes the purines normally present in FBS.

All other cell lines were obtained from American Type Cell Culture (Manassas, VA) and were cultured under corresponding standard culture conditions, according to the protocol recommended by the supplier.

Cells were typically grown using ˜1 to 2×104 cells per well at passage 3 to 15 suspended in 50 μl of the corresponding culture medium in the biosensor microplate, and were cultured at 37° C. under air/5% CO₂ for ˜1 day. The confluency for all cells at the time of assays was ˜100%.

iii. Optical Biosensor System and Cell Assays

Epic® wavelength interrogation system (Corning Inc., Corning, N.Y.) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of ˜15sec. The compound solutions were introduced by using the on-board liquid handling unit (i.e., pippetting).

The RWG biosensor is capable of detecting minute changes in local index of refraction near the sensor surface. Since the local index of refraction within a cell is a function of density and its distribution of biomass (e.g., proteins, molecular complexes), the biosensor exploits its evanescent wave to non-invasively detect ligand-induced dynamic mass redistribution in native cells. The evanescent wave extends into the cells and exponentially decays over distance, leading to a characteristic sensing volume of ˜150 nm, implying that any optical response mediated through the receptor activation only represents an average over the portion of the cell that the evanescent wave is sampling. The aggregation of many cellular events downstream the receptor activation determines the kinetics and amplitudes of a ligand-induced DMR.

For biosensor cellular assays, compound solutions were made by diluting the stored concentrated solutions with the HBSS (1× Hanks balanced salt solution, plus 20 mM Hepes, pH 7.1), and transferred into a 384 well polypropylene compound storage plate to prepare a compound source plate. Two compound source plates were made separately when a two-step assay was performed. In parallel, the cells were washed twice with the HBSS and maintained in 30 μl of the HBSS to prepare a cell assay plate. Both the cell assay plate and the compound source plate(s) were then incubated in the hotel of the reader system. After incubation the baseline wavelengths of all biosensors in the cell assay microplate were recorded and normalized to zero. Afterwards, a 2 to 10 min continuous recording was carried out to establish a baseline, and to ensure that the cells reached a steady state. Cellular responses were then triggered by transferring 10 μl of the compound solutions into the cell assay plate using the on-board liquid handler.

All studies were carried out at a controlled temperature (28° C.). At least two independent sets of experiments, each with at least three replicates, were performed. The assay coefficient of variation was found to be <10%.

### 2. Example 1

### The Influence of Cell Synchronization on CK2 Inhibitor-Triggered Responses in HeLa Cells

The de novo purine biosynthetic pathway produces purines which represent the building blocks for DNA and RNA synthesis, provide energy in chemical and redox reactions, and act as signaling molecules in regulatory pathways. The de novo purine pathway consists of ten stepwise reactions that server to convert phosphoribosyl pyrophosphate to inosine monophosphate. In general, prokaryotes tend to use freestanding single-functional enzymes for the chemical transformation, while the higher eukaryotes rely on multifunctional enzymes in this pathway. Using confocal fluorescence imaging and transfection techniques, Benkovic and his colleagues (An, S., et al. Science 2008, 320, 103-106) found that all of these enzymes act within a multi-enzyme complex framework, and form a purinosome complex. Such purinosome complexes are dynamic and reversible, dependent on cellular conditions. Since label-free optical biosensor is sensitive to mass redistribution, the dynamic process of purinosome formation and disassembly could be directly monitored using the biosensor such as Epic® system. Also, since CK2 could play an important role in regulating purine synthesis pathway, therefore CK2 inhibitors can be useful for detecting the dynamics of purinosome formation and disassembly.

Purinsome complexes are dynamic and sensitive to cellular status, therefore the influence of cell synchronization was examined first. The protocol for label-free cellular assays was modified based on the protocol used by Benkovic and his colleagues (An, S., et al. Science 2008, 320, 103-106). First, instead of engineered HeLa cells used by Benkovic et al., native HeLa cells (without any genetic alterations) were used. Second, instead of low confluent cells, highly confluent monolayer cells were used. Third, instead of monitoring fluorescent clusters in live cells, an integrated DMR signal was recorded using Epic® system. Fourth, instead of purine depleted medium, a regular serum containing medium was used. Results showed that once cultured in regular serum medium, HeLa cells reached high confluency and formed a monolayer on Epic® biosensor microplate surface, when the initial seeding density of →10K per well (FIG. 2B, FIG. 2C). When the seeding density is relatively low (e.g., 5K per well), the cells reached a confluency of ˜70% 1 day after culture (FIG. 2A).

The two known CK2 inhibitors, TBB and DMAT, triggered detectable DMR signals in HeLa cells cultured using three different seeding densities using the regular serum medium. However, the TBB DMR signal is dramatically sensitive to cellular status (FIG. 2D), while the DMAT DMR signal seems insensitive to cellular status (FIG. 2E). These results indicate that in un-stimulated cells, there are certain basal purinosome complexes which are cellular status dependent. There are fewer basal purinosome complexes in proliferating cells obtained using low seeding density compared to quiescent cells obtained using high seeding density. As a result, both TBB and DMAT promote the formation of purinosomes in proliferating cells. However, in quiescent cells obtained using high seeding density, TBB causes the disassembly of basal purinosome complexes, but DMAT promotes the further formation of purinosome complexes. This result is unexpected because it is reported in literature that purine depletion is required for purinosome formation (An, S., et al. Science 2008, 320, 103-106).

To distinguish the possible mechanism, HeLa cells were prepared using a protocol similar to the one used in Benkovic and this colleagues; i.e., HeLa cells were cultured using a purine depleted medium until they reached desired confluency. Results showed that both TBB and DMAT led to similar positive DMR (P-DMR) signals, indicating that under purine depleted culture conditions, both CK2 inhibitors promoted the formation of purinosome complexes when cells are at high confluency. Most interestingly, both CK2 inhibitor-induced DMR signals are insensitive to cellular confluency, but gave rise to significant assay variations when the seeding numbers are low.

Studies using transit transfection of fluorescent enzymes involving the purine synthesis showed that TBB triggered disassembly of purinsome complexes, while DMAT led to formation of purinosome complexes in these pruine depleted medium culture cells, when cells were at low confluency (˜50%) (data not shown). The inconsistency in the action of TBB between fluorescence imaging and label-free cellular assays can be due to that confocal fluorescence imaging is largely single cell-based, while label-free assay is largely population cells-based, as well as different confluencies used in both assays.

### 3. Example 2

### CK2 Inhibitor-Induced DMR Signals in HeLa Cells are Dynamic and Reversible

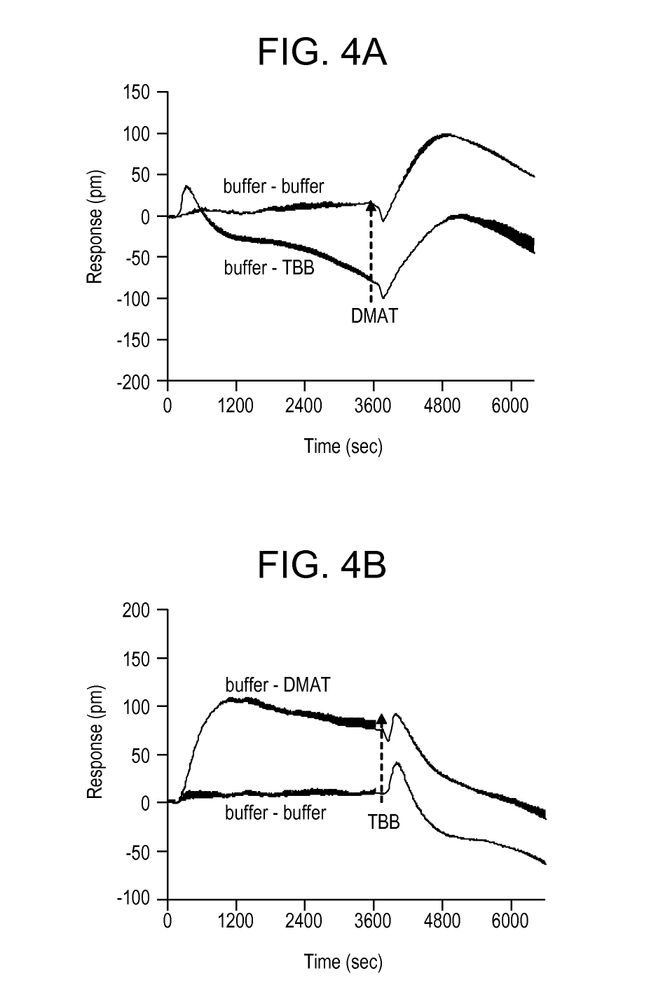

Since HeLa cells obtained using high initial seeding numbers of cells under regular serum culture medium responded to the two CK2 inhibitors differently, and both inhibitors led to robust DMR signals in the synchronized HeLa cells, the dynamics of DMR signals was examined. Results were summarized in FIG. 4. Here the cells were pre-treated with the assay vehicle only (i.e., buffer), followed by TBB and DMAR in a sequential order. Each step lasts about 1 hr. Results show that HeLa cells first respond to TBB with a N-DMR signal; and the pretreatment of cells with TBB did not alter the kinetics of the DMAT response, but slightly potentiated the DMAT response (FIG. 4A). However, when HeLa cells were first simulated with DMAT the cells responded with a P-DMR signal, and the DMAT-treated cells further responded to TBB with N-DMR similar to the buffer treated cells (FIG. 4B). These results indicate that both CK2 inhibitors trigger a dynamic and reversible DMR signals in HeLa cells, and the two inhibitors display different modes of action.

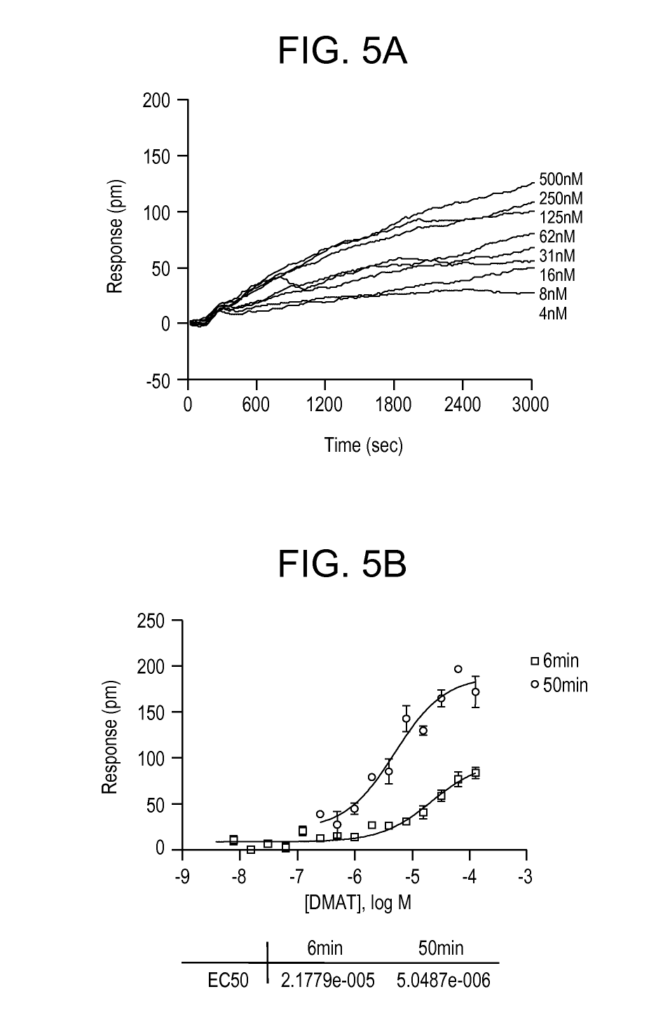

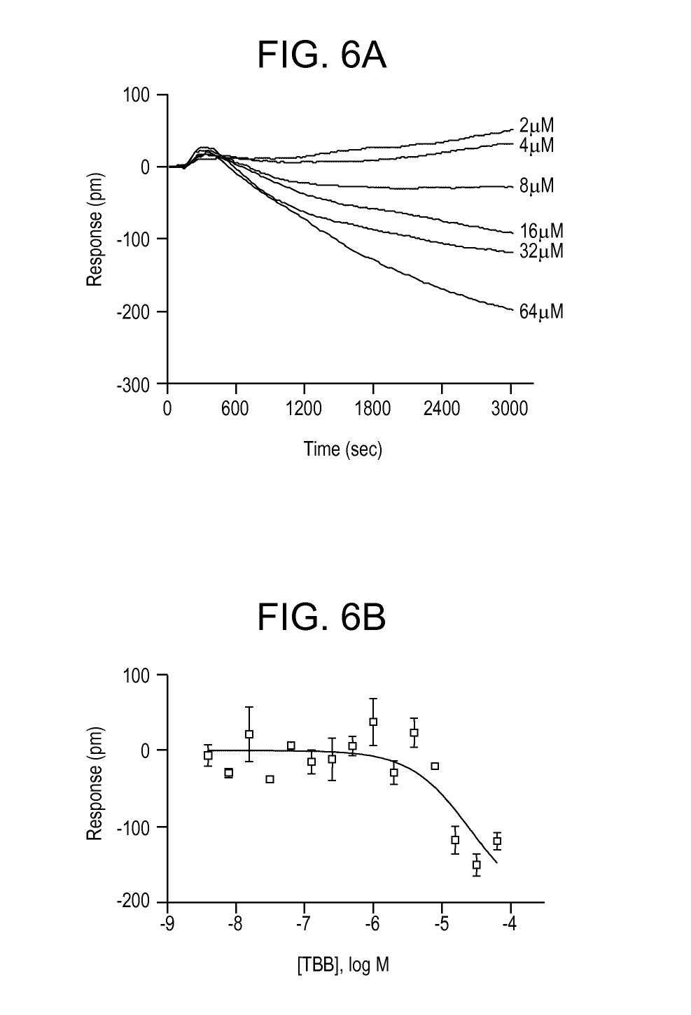

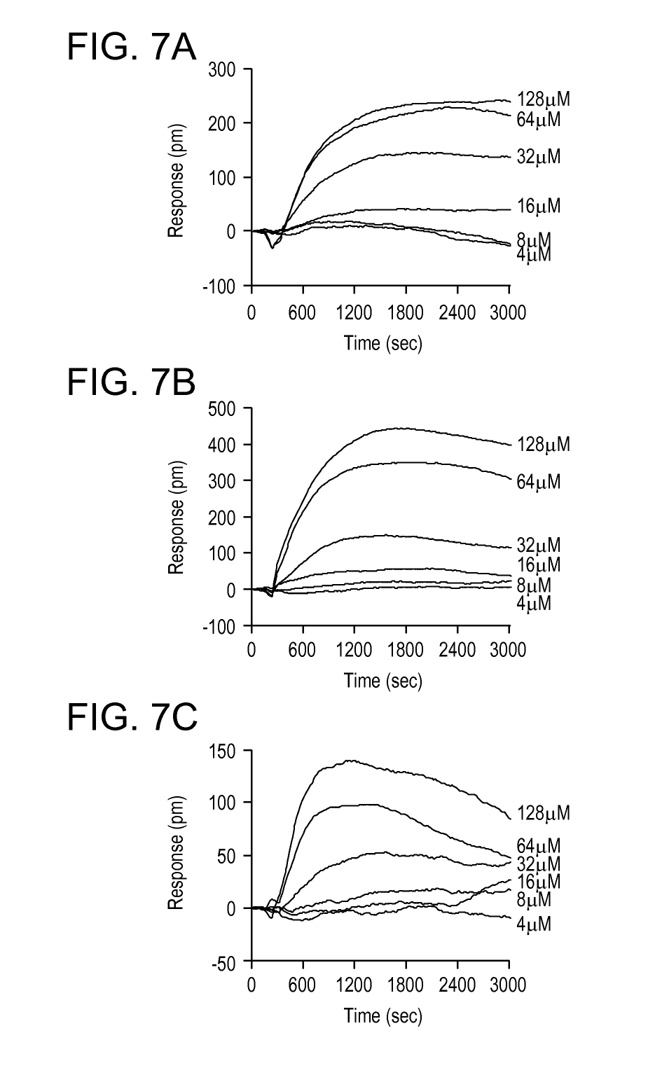

Detailed pharmacology studies indicate that both inhibitors triggered DMR signals are saturable (FIG. 5 and FIG. 6). DMAT resulted in a dose-dependent and saturable P-DMR response in the synchronized cells, leading to an EC₅₀ of ˜5 to 22 μM, dependent on the time points after the DMAT stimulation (FIG. 5). Similarly three other CK2 inhibitors, apigenin, DRB, and TBCA, led to DMR signals similar to the DMAT response (FIG. 7). On the other hand, TBB also led to a dose-dependent and saturable DMR signal, but in the opposite direction (i.e., N-DMR), with an EC50 of 25 μM (FIG. 6). Furthermore, the cells pretreated with TBCA or DRB became desensitized to the sequential DMAT stimulation, but only slightly potentiated the TBB response (data not shown). Taken together, these results indicate that label-free biosensor cellular assays are feasible to detect the CK2 activity in live cells, and can classify CK2 inhibitors into two types: purinosome promoting CK2 inhibitors (e.g., DMAT, apigenin, DRB, TBCA), and pruinosome disrupting CK2 inhibitors (e.g., TBB).

### 4. Example 3

### The Dynamics of CK2 Inhibitor-Induced DMR Signals is Sensitive to Microfilament Remodeling

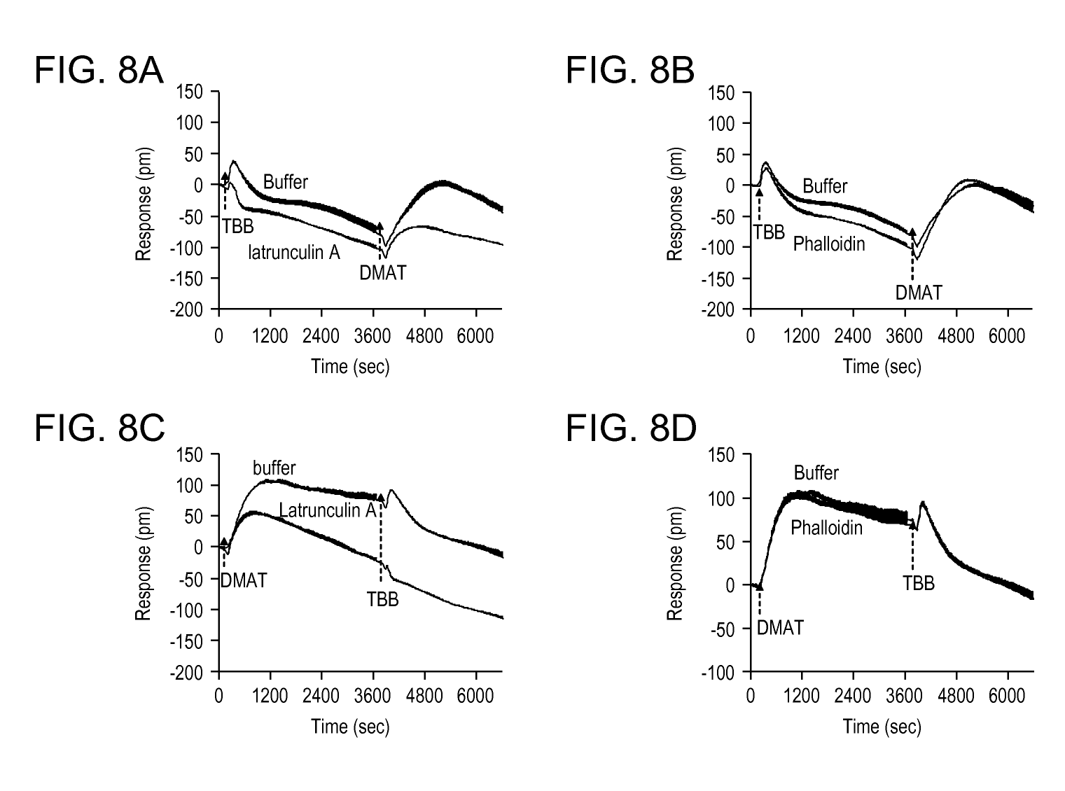

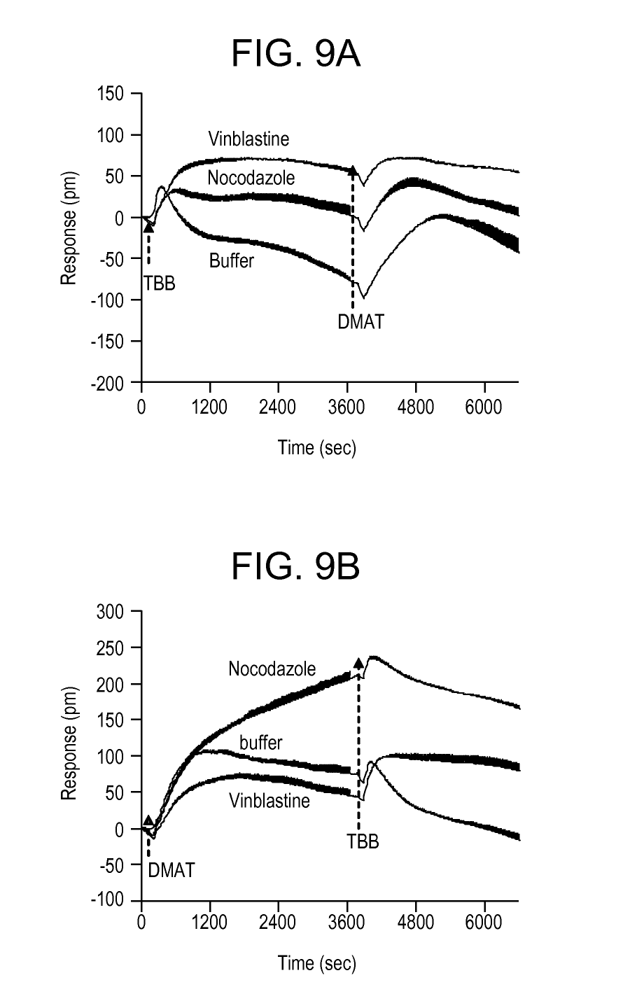

Example 2 showed that DMR signals induced by a CK2 inhibitor are dynamic, and that a DMR triggered by a purinosome promoting CK2 inhibitor can be reversed by the sequential stimulation with a purinosome disrupting CK2 inhibitor, and vice versa. Thus, the dynamics of the TBB and DMAT DMR signals can be used as an indicator to study the cellular process and signaling pathway linked to CK2 inhibitor regulated purinosome process. The impacts of different microfilament modulators were studied because microfilaments are central to cellular signaling and functions. The results are summarized in FIGS. 8 and 9.

As shown in FIG. 8A, the actin disruption agent latrunculin A alters the TBB response, by completely inhibiting the early DMR event of the TBB response. The latrunculin A-TBB treated cells responded with a smaller DMAT response to the sequential DAMT stimulation, compared to the buffer-TBB treated cells. Similarly, the latrunculin A pretreatment attenuated the first DMAT response, and also altered the sequential TBB response (FIG. 8C). On the other hand, the actin polymerization promoting agent phalloidin had little impact on the dynamics and kinetics of either TBB or DMAT responses, regardless of the sequential order (FIG. 8B and FIG. 8D). Taken together, these results indicate that actin remodeling is required to both formation and disassembly of the purinosome complexes.

As shown in FIG. 9, the microtubule modulators impacts the dynamics of both inhibitors induced DMR signals. Both microtubule disrupting agents vinblastine and nocodazole reversed the TBB response, and caused the suppression of the sequential DMAT response (FIG. 9A). Similar impact was observed for vinblastine when the dynamics is monitored in a reversed sequential order (instead of TBB-DMAT, the order is DMAT and then TBB) (FIG. 9B). However, nocodazole potentiated the DMAT response, and only slightly suppressed the sequential TBB response. Taken together, there is difference in mechanism to alter microtubule structure by the two agents, microtubule remodeling is involved in CK2 inhibitors induced DMR signals.

### 5. Example 4

### The CK2 Inhibitors Induced DMR Signal is Sensitive to the Activation of Endogenous Gi-Coupled Receptors

The linkage between GPCR signaling and CK2-induced regulated purinosome dynamics was examined. First, the expressions of endogenous GPCRs are examined using RT-quantitive PCR. Results showed that HeLa cells endogenously express alpha2A adrenergic receptor, beta2-adrenegic receptor, S1P1, S1P2 and S1P4 receptors, CB1 receptors, and LPA1, LPA2, and LPAS receptors (data not shown).

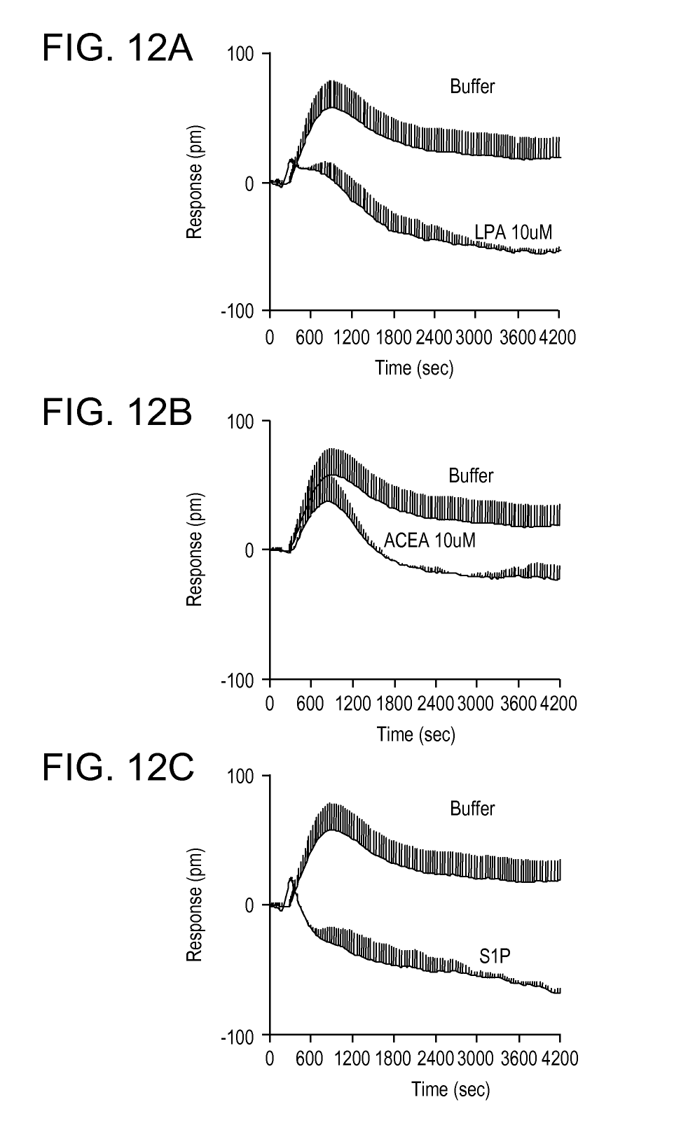

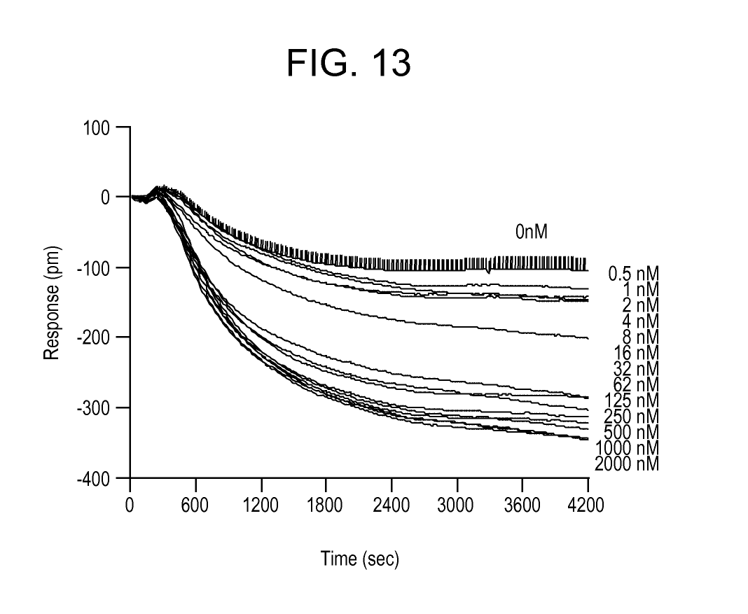

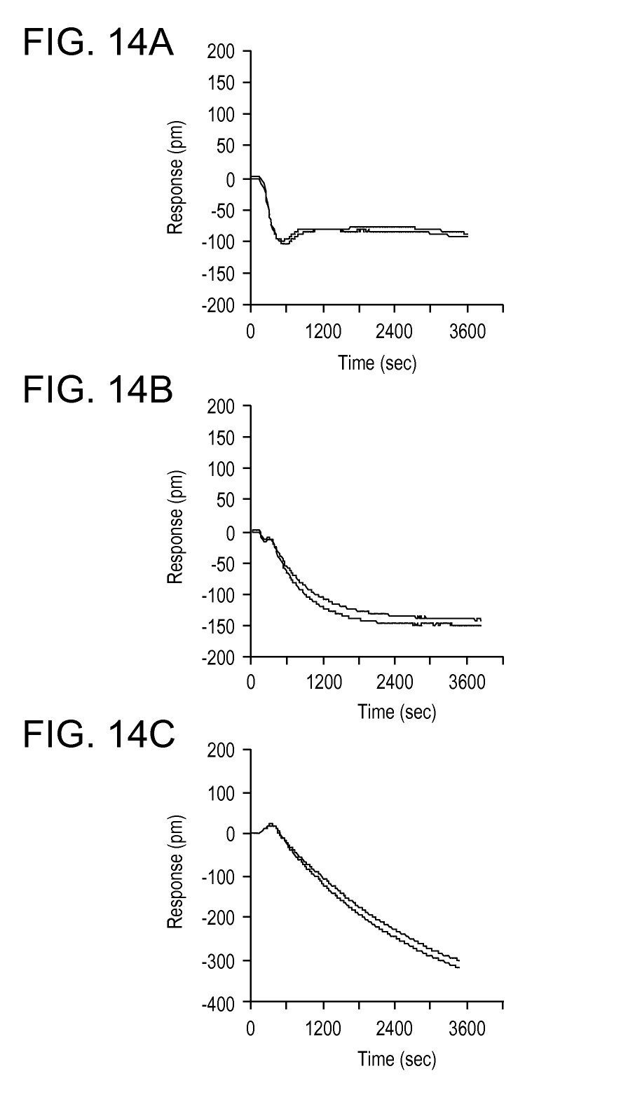

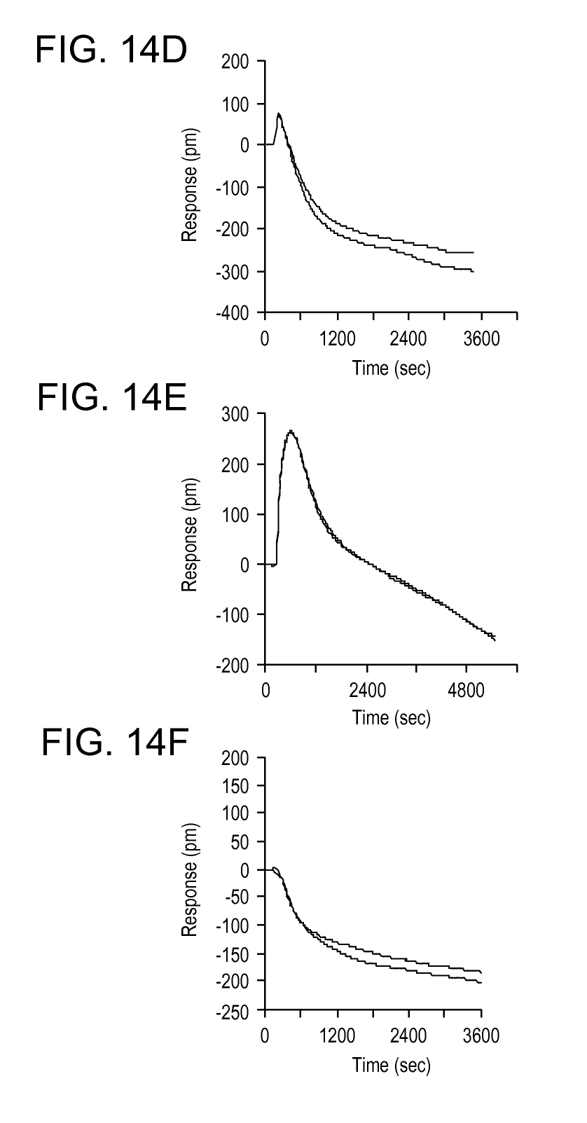

As expected, the Gi-coupled alpha2A adrenergic receptor agonist oxymetazoline led to a Gi-like DMR signal in HeLa cells (FIG. 10A). The oxymetazoline treated cells responded to the sequential stimulation with DMAT and TBB, respectively FIGS. 10B and C). The oxymetazoline pretreatment attenuated the DMAT response (FIG. 10B). The oxymetazoline-DMAT treated cells caused potentiation of the TBB response (FIG. 10C). Similar results were observed to three other Gi-coupled receptor agonists LPA, ACEA, and S1P, respectively (FIG. 12A to C). Alpha2A receptor is a Gi-coupled receptor, while LPA receptors, S1P receptors and CB1 receptors are also Gi-coupled receptors. LPA is the natural agonist of LPA receptors, S1P is the natural agonist of S1P receptors, while ACEA is a CB1 selective agonist. The impact of alpha2A agonists (e.g., oxymetazoline and clonidine) on the dynamics of CK2 inhibitors induced DMR signals is dose-dependent (FIG. 12).

On the other hand, the Gs-coupled beta2 adrenergic receptor agonist terbutaline triggered a Gs-like DMR signal with a maximal response that is much smaller than the oxymetazoline response (FIG. 11A). The terbutaline pretreatment had little or no impact on the dynamics of DMAT-TBB responses (FIGS. 11B and C). Taken together, these results indicate that the activation of the Gi-coupled receptors, but not the Gs-coupled receptors (e.g., beta2AR), is linked to the formation and disassembly process of purinosome complexes. The most possible mechanism is that the activation of Gi-coupled receptors, but not Gs-coupled receptors, results in an increase in purinosome complexes formed.

### 6. Example 5

### The CK2 Inhibitors Induced DMR Signals are Universal to Several Cell Lines Examined

Since de novo purine synthesis is central to all cells, the CK2 inhibitor induced

DMR signals related to purinosome dynamics were examined across a panel of cancers including skin cancer (cell line A431), lung cancer (cell line A549), normal human embryonic kidney (cell line HEK293), breast cancer (cell line MDA-AB-231), colon cancer (cell line HT29) and prostate cancer (cell line PC3). Results showed that among all cells examined, the purinosome disrupting CK2 inhibitor TBB led to similar DMR signals, except for HT29 cells wherein the TBB DMR signal consists of two phases—an initial P-DMR followed by a N-DMR. Nonetheless, these results indicate that CK2 inhibitors-regulated purinosome dynamics is universal across different types of cells. Since purine synthesis pathway inhibitors represent proven strategy for anti-cancer agents, and the pruinosome formation is important for de novo purine synthesis, purinosome disrupting CK2 inhibitors could act as effective purine synthesis pathway inhibitors, thus represent novel approach for anti-cancer treatment. These purinsome-disrupting CK2 inhibitors can be used alone or combinations with other anti-cancer agents to increase therapeutic potential and efficacy.

We claim:

1. A method for testing a molecule comprising,
a) incubating the molecule with a cell system comprising a multienzyme complex forming an incubated cell system,
b) assaying the incubated cell system with a label free biosensor system.
c) measuring the ability of the molecule to modulate a the multi-enzyme complex.

2. The method of claim 1, further comprising the step of classifying the molecule as a multienzyme complex modulator.

3. The method of claim 1, wherein the cell system comprises synchronized cells.

4. The method of claim 3, where the synchronization of the cells comprises culturing the cells in a purine rich serum medium with a high initial seeding number such that the cells once attached reach high confluency; culturing cells in serum but purine depleted medium; or culturing the cells of a highly confluency under long term starvation conditions.

5. The method of claim 4, wherein the cells are synchronized to have a basal level of multienzyme complex activity.

6. The method of claim 2, wherein the multienzyme complex modulator comprises a multienzyme disassembly promoting agent.

7. The method of claim 6, wherein the multienzyme disassembly promoting agent comprises a purinosome complex disrupting agent.

8. The method of claim 7, wherein the purinosome complex disrupting agent comprises a purinosome disrupting CK2 inhibitor.

9. The method of claim 8, wherein the purinosome disrupting CK2 inhibitor comprises TBB.

10. The method of claim 8, wherein the purinosome disrupting CK2 inhibitor is a purine synthesis pathway inhibitor.

11. The method of claim 2, wherein the multienzyme complex modulator comprises a multienzyme complex promoting agent.

12. The method of claim 11, wherein the mutlienzyme complex promoting agent comprises a purinosome complex promoting agent.

13. The method of claim 12, wherein the purinosome complex promoting agent comprises a purinosome promoting CK2 inhibitor.

14. The method of claim 13, wherein the purinosome promoting CK2 inhibitor comprises DMAT, apigenin, DRB, or TBCA.

15. The method of claim 1, wherein the multienzyme complex is involved in purine synthesis.

16. The method of claim 1, wherein the cell system comprises live cells having CK2 activity.

17. The method of claim 1, further comprising the step of incubating the cells with a reference probe.

18. The method of claim 17, wherein the reference probe comprises a purinsome complex promoting agent.

19. The method of claim 17, wherein the purinosome complex promoting agent comprises DMAT, apigenin, DRB, or TBCA.

20. The method of claim 17, wherein the reference probe comprises a purinosome complex disrupting agent.

21. The method of claim 20, wherein the purinosome complex promoting agent comprises TBB.

22. The method of claim 2, wherein the step of classifying comprises comparing a molecule induced biosensor response with a reference probe biosensor response wherein the similarity in the biosensor responses is an indicator that the molecule is classified in the same class as the reference probe.

23. The method of claim 22, wherein the reference probe comprises a CK2 inhibitor.

24. The method of claim 22, wherein the biosensor response comprises a DMR signal.

25. The method of claim 2, wherein the step of comparing comprises assaying the molecule against both a purinosome complex disrupting agent and a purinosome promoting agent.

26. The method of claim 25 wherein the purinosome complex disrupting agent comprises TBB and the purinosome promoting agent comprises DMAT.

27. The method of claim 25, wherein a molecule that results in a DMR signal similar to TBB, and selectively inhibits the TBB DMR signal but potentiates the DMAT DMR signal is a purinosome disrupting agent.

28. The method of claim 25, wherein a molecule that results in a DMR signal similar to DMAT, and selectively inhibits the DMAT DMR signal but potentiates the TBB DMR signal is a purinosome promoting agent.

29. The method of claim 22, wherein the step of comparing comprises comparing a molecule-induced DMR index with a known CK2 inhibitor DMR index acting on a panel of CK2 inhibitor sensitive cells, wherein the similarity between the two indices is an indicator that the molecule is a CK2 inhibitor.

30. The method of claim 2, wherein the step of classifying comprises generating DMR modulation indices of both a molecule and a known CK2 inhibitor against a panel of cells/markers, wherein the similarity between the two indices is an indicator that the molecule is a CK2 inhibitor.

31. The method of claim 2, further comprising assaying the sensitivity of the molecule induced DMR signal to the preactivation of an endogenous Gi-coupled receptor.

32. The method of claim 27, wherein the molecule induced DMR that is similar to TBB, and is potentiated in the cell system having the preactivaction of the Gi-coupled receptor is an indicator that the molecule is purinosome disrupting CK2 inhibitor.

33. A method of treating cancer in a subject comprising administering a purinosome disrupting CK2 inhibitor.

34. The method of claim 33, wherein the cancer is leukemia, colorectal cancer, prostate cancer, breast cancer, and lymphoma, squamous cell carcinoma of head and neck, lung, brain glioma cancer or a cancer phenotype related to abnormal activity of CK2 kinase.

35. The method of claim 33, wherein the purinosome disrupting CK2 inhibitor comprises a pharmaceutically acceptable salt of the purinsome disrupting CK2 inhibitor.

36. A method of treating a varial infection in a subject comprising administering a purinosome disrupting CK2 inhibitor.

37. The method of claim 36, wherein the viral infection comprises a Herpes virus or Cytomegalovirus.

38. A method of treating an inflammation condition in a subject comprising administering a purinosome disrupting CK2 inhibitor.

39. The method of claim 38, wherein the inflammatory condition comprises a chronic inflammation disorder related to abnormal activity of CK2 kinase.

40. The method of claim 39, wherein the chronic inflammation disorder is an intestinal disorder or glomerulonephritis.

41. The method of claim 40, wherein the intestinal disorder is inflammatory bowel disease, ulcerative colitis, or Crohn's disease.

42. The method of claim 38, wherein the composition is administered in a therapeutically effective amount of the purinosome disrupting CK2 inhibitor.

43. The method of claim 42, wherein the subject is in need of treatment with the purinosome disrupting CK2 inhibitor.
